(19) United States
(12) Patent Application Publication
(10) Pub. No.: US 2022/0136116 A1
(43) Pub. Date: May 5, 2022

(54) HYPOCHLOROUS ACID REFILL GENERATOR METHOD AND DEVICES

(71) Applicant: JOE GANAHL, Honolulu, HI (US)

(72) Inventor: JOE GANAHL, Honolulu, HI (US)

(21) Appl. No.: 17/120,166

(22) Filed: Dec. 12, 2020

Related U.S. Application Data
(63) Continuation-in-part of application No. 17/084,611, filed on Oct. 29, 2020.

(51) Int. Cl. C25B 1/26; C02F 1/32; B01F 3/12; B01F 15/00; B01F 13/00; C25B 1/50; C25B 9/17; C25B 9/65; C25B 15/029; C25B 15/08; A61L 2/18

(52) U.S. Cl. CPC C25B 1/26; B01F 2215/008; B01F 3/1221; B01F 15/00376; B01F 13/0022; B01F 13/0033; C25B 1/50; C25B 9/17; C25B 9/65; C25B 15/029; C25B 15/08; A61L 2/18; C02F 2209/40; C02F 2201/3222; C02F 1/32

(57) ABSTRACT

The embodiments disclose an apparatus including at least one hypochlorous acid generator for producing purified hypochlorous acid from purified water and pure salt, a mixing tank container hypochlorous acid generator for processing the purified water and pure salt, a cap with vent configured to release gases created during an electrolysis operation, a water intake port to fill the mixing tank container with fill water automatically using an automatic system intake valve, a water drain port to drain liquid from the mixing tank container, an AC port to route external power circuits connections, at least one crossing double electrode module configured to provide ultraviolet light to purify water and perform electrolysis, an LCD control panel coupled to control buttons to display processing status and operation control settings, and a hypochlorous acid generator app on a user digital device to transmit hypochlorous acid generator control settings.

### CROSS REFERENCE TO RELATED APPLICATION

The present application is a continuation-in-part made under 35 U.S.C. § 119(e) to currently pending non-provisional patent application Ser. No. 17/084,611, titled “CONTAINER WITH HEATING/COOLING ASSEMBLY AND REMOVABLE POWER SOURCE MODULES” having a filing date of Oct. 29, 2020, by Ganahl.

### BACKGROUND OF THE INVENTION

Sanitizing surfaces that are contacted by persons is a key to reducing and even eliminating the spread of infections caused by bacteria, germs, fungi and viruses including Sars-Cov-2 that causes Covid-19. One method to disinfect surfaces is the application of hypochlorous acid (HOCL). The greater the number of surfaces treated the lesser the chance of spreading for example Covid-19. The larger the area of a home, office, retail store, commercial or industrial area the greater the volume of HOCL needed to treat the surfaces. This can also require a greater amount of time due to wait times for a HOCL device to make an additional fresh viable batch of HOCL. The greater amount of time having to be spent can cause short cuts that may lead to not total coverage and thorough disinfecting all the surfaces increasing the opportunity of someone getting infected.

### BRIEF DESCRIPTION OF THE DRAWINGS

FIG. 1A is a front elevation view of the container as described in accordance with at least one embodiment of the present invention.

FIG. 1B is a right side elevation view of the container illustrated in FIG. 1A.

FIG. 2A is a front elevation view of the main body portion and cap as disclosed in accordance with at least one embodiment of the present invention.

FIG. 2B is a right side elevation view of the main body portion and cap illustrated in FIG. 2A.

FIG. 2C is a right side elevation cut-away view of the main body portion and cap illustrated in FIGS. 2A and 2B.

FIG. 2D is a bottom perspective view of the main body portion as disclosed in accordance with at least one embodiment of the present invention.

FIG. 3A is a perspective view of the heating assembly as disclosed in accordance with at least one embodiment of the present invention.

FIG. 3B is a front elevation view of the heating assembly as disclosed in accordance with at least one embodiment of the present invention.

FIG. 3C is a right side elevation view of the heating assembly illustrated in FIG. 3B.

FIG. 3D is a cut away view of the heating assembly illustrated in FIGS. 3B and 3C.

FIG. 3E is a bottom view of the heating assembly illustrated in FIGS. 3B and 3C.

FIG. 3F is a top view of the heating assembly illustrated in FIGS. 3B and 3C.

FIG. 4A is an exploded view of the heating assembly and main body portion as disclosed in accordance with at least one embodiment of the present invention.

FIG. 4B is a left side elevation view of the main body portion, heating assembly and cap as disclosed in accordance with at least one embodiment of the present invention.

FIG. 4C is a rear elevation view of the embodiment illustrated in FIG. 4B.

FIG. 4D is a cut away view of the embodiment illustrated in FIGS. 4B and 4C.

FIG. 5A is a front elevation view of the battery module as disclosed in accordance with at least one embodiment of the present invention.

FIG. 5B is a right side elevation view of the battery module as disclosed in accordance with at least one embodiment of the present invention.

FIG. 5C is a top view of the battery module as disclosed in accordance with at least one embodiment of the present invention.

FIG. 5D is a perspective view of the battery module as disclosed in accordance with at least one embodiment of the present invention.

FIG. 5E is an exploded view of the battery module as disclosed in accordance with at least one embodiment of the present invention.

FIG. 5F is a cut away view of the battery module as disclosed in accordance with at least one embodiment of the present invention.

FIG. 5G is a cut away view of the container as disclosed in accordance with at least one embodiment of the present invention, including the main body portion, heating assembly, battery module and cap.

FIG. 6A is an elevation view of an exemplary power source module as disclosed in accordance with at least one embodiment of the present invention.

FIG. 6B is a top view of the exemplary power source module illustrated in FIG. 6A.

FIG. 6C is a cut away view of the exemplary power source module illustrated in FIG. 6A.

FIG. 6D is a front elevation view of the container as disclosed in accordance with at least one embodiment of the present invention, including the main body portion, heating assembly, exemplary power source module and cap.

FIG. 6E is a left side elevation view of the embodiment illustrated in FIG. 6D.

FIG. 6F is a left side cut away view of the embodiment illustrated in FIG. 6D.

FIG. 7A is an elevation view of another exemplary power source module as disclosed in accordance with at least one embodiment of the present invention.

FIG. 7B is a top view of the exemplary power source module illustrated in FIG. 7A.

FIG. 7C is a cut away view of the exemplary power source module illustrated in FIG. 7A.

FIG. 7D is a left side elevation view of the container as disclosed in accordance with at least one embodiment of the present invention, including the main body portion, heating assembly, exemplary power source module and cap.

FIG. 7E is a rear elevation view of the embodiment illustrated in FIG. 7D.

FIG. 7F is a left side cut away view of the embodiment illustrated in FIG. 7D.

FIG. 8 shows for illustrative purposes only an example of a collapsible pump assembly for water purification applications at outdoor remote locations of one embodiment.

FIG. 9 shows for illustrative purposes only an example of water purification modules of one embodiment.

FIG. 10A shows for illustrative purposes only an example of a UV cap module of one embodiment.

FIG. 10B shows for illustrative purposes only an example of a UV cap module cross section of one embodiment.

FIG. 10C shows for illustrative purposes only an example of a UV cap module radiating ultraviolet light disinfecting the top section of the collapsible pump assembly of one embodiment.

FIG. 10D shows for illustrative purposes only an example of a UV cap module radiating ultraviolet light disinfecting the bottom section of the collapsible pump assembly of one embodiment.

FIG. 11A shows for illustrative purposes only an example of a filter box module of one embodiment.

FIG. 11B shows for illustrative purposes only an example of a filter mesh element of one embodiment.

FIG. 11C shows for illustrative purposes only an example of a collapsible pump assembly extended of one embodiment.

FIG. 11D shows for illustrative purposes only an example of a collapsible pump assembly compressed of one embodiment.

FIG. 12A shows for illustrative purposes only an example of a first crossing double electrode module of one embodiment.

FIG. 12B shows for illustrative purposes only an example of a second crossing double electrode module of one embodiment.

FIG. 12C shows for illustrative purposes only an example of a portable HOCL generator assembly of one embodiment.

FIG. 13 shows for illustrative purposes only an example of filtering water for generating HOCL of one embodiment.

FIG. 14 shows for illustrative purposes only an example of depositing a pure salt into the filtered water of one embodiment.

FIG. 15 shows for illustrative purposes only an example of an electrolysis process of one embodiment.

FIG. 16 shows for illustrative purposes only an example of a portable HOCL generator sprayer of one embodiment.

FIG. 17 shows for illustrative purposes only an example of a portable HOCL generator app of one embodiment.

FIG. 18A shows for illustrative purposes only an example of a filter box housing of one embodiment.

FIG. 18B shows for illustrative purposes only an example of a coffee filter of one embodiment.

FIG. 18C shows for illustrative purposes only an example of preloaded cup of one embodiment.

FIG. 18D shows for illustrative purposes only an example of a coffee ground blend of one embodiment.

FIG. 19 shows a block diagram of an overview of collapsible pump assembly coffee brewing of one embodiment.

FIG. 20 shows for illustrative purposes only an example of a first HOCL generator of one embodiment.

FIG. 21 shows for illustrative purposes only an example of a first HOCL Generator transparent view of one embodiment.

FIG. 22A shows for illustrative purposes only an example of a second HOCL generator front right side prospective view of one embodiment.

FIG. 22B shows for illustrative purposes only an example of a second HOCL generator rear left side prospective view of one embodiment.

FIG. 23A shows for illustrative purposes only an example of a mixing tank container translucent prospective view of one embodiment.

FIG. 23B shows for illustrative purposes only an example of a second HOCL generator front view of one embodiment.

FIG. 24A shows for illustrative purposes only an example of a cross blade electrode module of one embodiment.

FIG. 24B shows for illustrative purposes only an example of an ultraviolet LED of one embodiment.

FIG. 24C shows for illustrative purposes only an example of a cross blade electrode module bottom view of one embodiment.

FIG. 25 shows a block diagram of an overview of a power current controller of one embodiment.

FIG. 26 shows for illustrative purposes only an example of a HOCL generator app of one embodiment.

### DETAILED DESCRIPTION OF THE INVENTION

In a following description, reference is made to the accompanying drawings, which form a part hereof, and in which is shown by way of illustration a specific example in which the invention may be practiced. It is to be understood that other embodiments may be utilized and structural changes may be made without departing from the scope of the embodiments.

### General Overview:

It should be noted that the descriptions that follow, for example, in terms of a hypochlorous acid refill generator method and devices is described for illustrative purposes and the underlying system can apply to any number and multiple types attachable and removable modules. In one embodiment of the present invention, the hypochlorous acid refill generator method and devices can be configured using an attachable battery base module. The hypochlorous acid refill generator method and devices can be configured to include phased pulsed current controls and can be configured to include AC external power source connections using the embodiments.

### DETAILED DESCRIPTION OF THE INVENTION

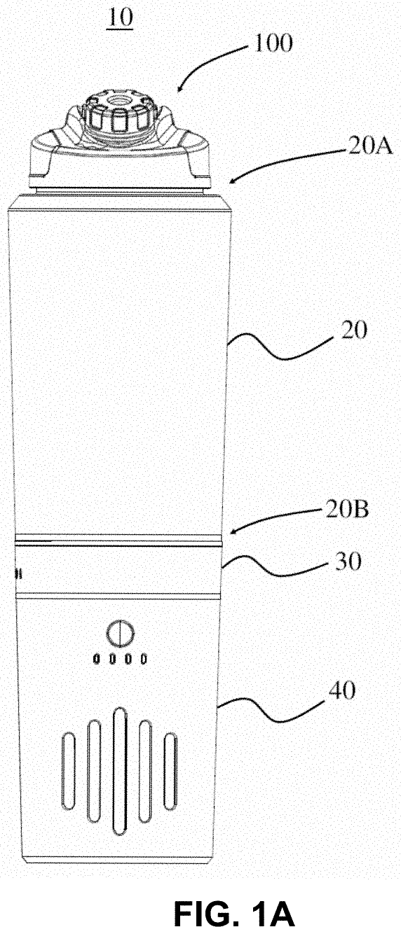

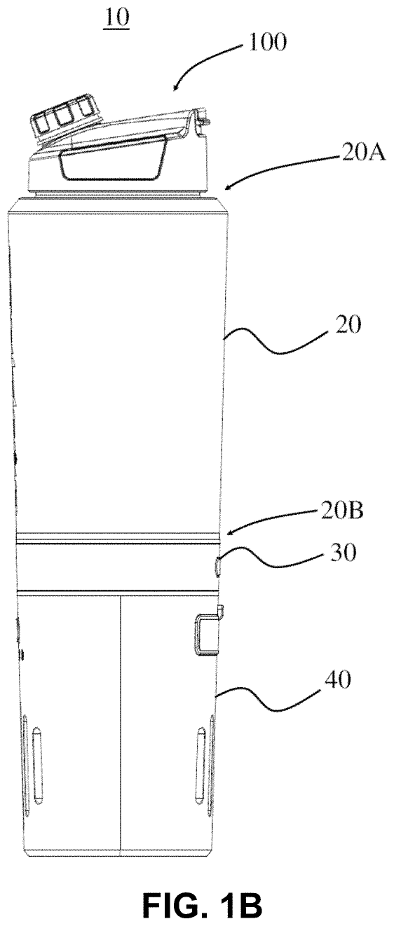

As shown in the accompanying drawings, and with particular reference to FIGS. 1A and 1B, the present invention is directed to a beverage container, generally referenced as 10. In particular, as described herein, the beverage container 10 of at least one embodiment is structured and configured to effectively and efficiently control the temperature of a beverage, liquid, fluid or other contents therein. For instance, the beverage container 10 of at least one embodiment includes a heating assembly 30 that is adapted to heat the contents of the container 10 to a predetermined or selected temperature range, and in some cases, at or above a boiling point (e.g., at least 212 degrees Fahrenheit). In some embodiments, the beverage container 10 and/or the heating assembly 30 thereof includes a plurality (e.g., three (3)) heating modes which, when selected, will function to maintain the temperature of the beverage or contents of the container 10 at corresponding predefined, user preselected or preset temperatures.

Accordingly, still referring to FIGS. 1A and 1B, the container 10 of at least one embodiment includes a main body portion, referenced as 20, and a heating/cooling assembly, referenced as 30. As shown, a cap 100 can be secured or connected to a top portion or top end 20A of the main body portion 20, with the heating assembly 30 connected or attached to the bottom end 20B. In this manner, the main body portion 20 is adapted to retain an amount of fluid or other contents therein, while the cap 100 can be used to selectively control the dispensing of the fluid therefrom.

The heating assembly 30 is structured and configured to provide controlled heat to the contents of the container 10 in order to control the temperature thereof, such as, for example, by heating the fluid or other contents to a selected temperature or temperature range. In some instances, the heating assembly 30 is powerful enough and/or specifically adapted to heat the fluid or other contents of the container 10 to a temperature at or above a boiling point. This allows a user to selectively boil the contents of the container 10 for an amount of time, if desired. As also shown in FIGS. 1A and 1B, one or more additional modules, generally referenced as 40, can be secured or selectively removable connected to container 10, such as, to the bottom of the heating assembly 30, for example, via a cooperatively constructed connection assembly, including, but in no way limited a twist and lock type of connection assembly.

The one or more modules 40, as shown in FIGS. 1A and 1B, can include a battery module, which includes one or more batteries (e.g. rechargeable lithium ion batteries, replaceable batteries, etc.) that function to provide power or electricity to the heating assembly 30. Other embodiments described and illustrated herein, may include modules 50, 60 with a power or electrical cable for connection to an external power source, such as an AC or DC power source. In this regard, the beverage container 10 may be connected to an external power source (e.g., electrical outlet in a home, office, or vehicle) in order to provide necessary power or electrical connections to the heating assembly 30. In this manner, the additional, auxiliary or attachment module(s) 40 may be electrically interconnected to the heating assembly 30 in order to implement the present invention in the intended manner.

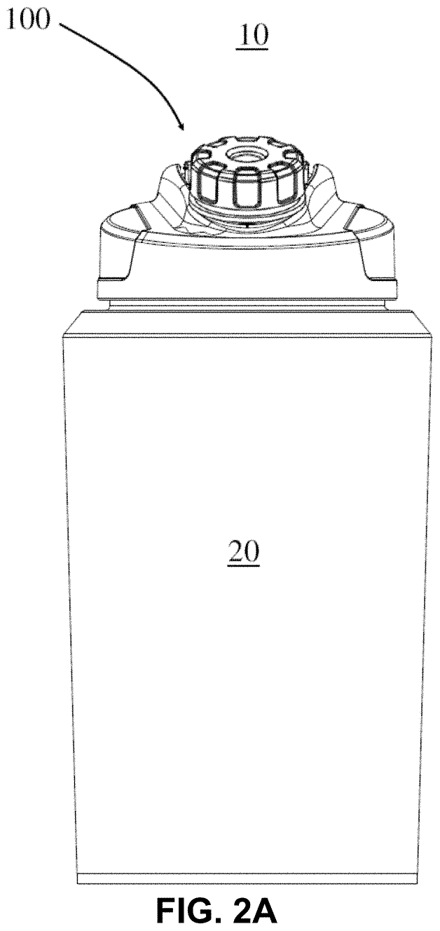

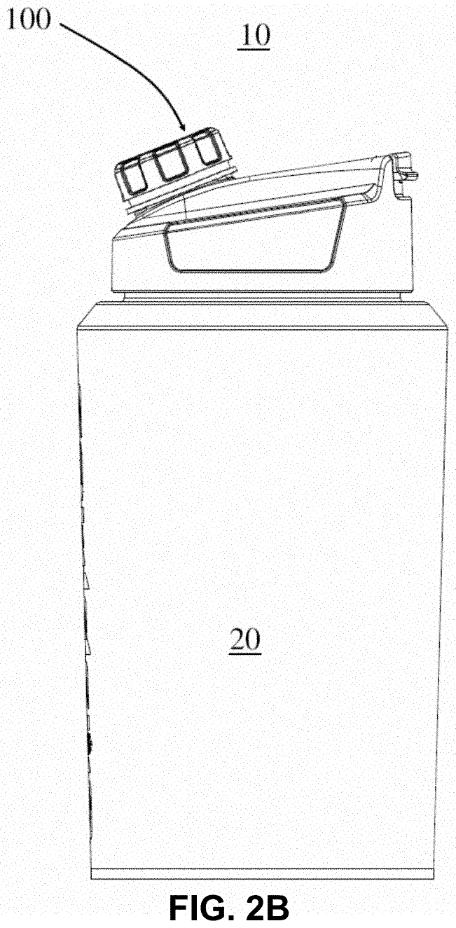

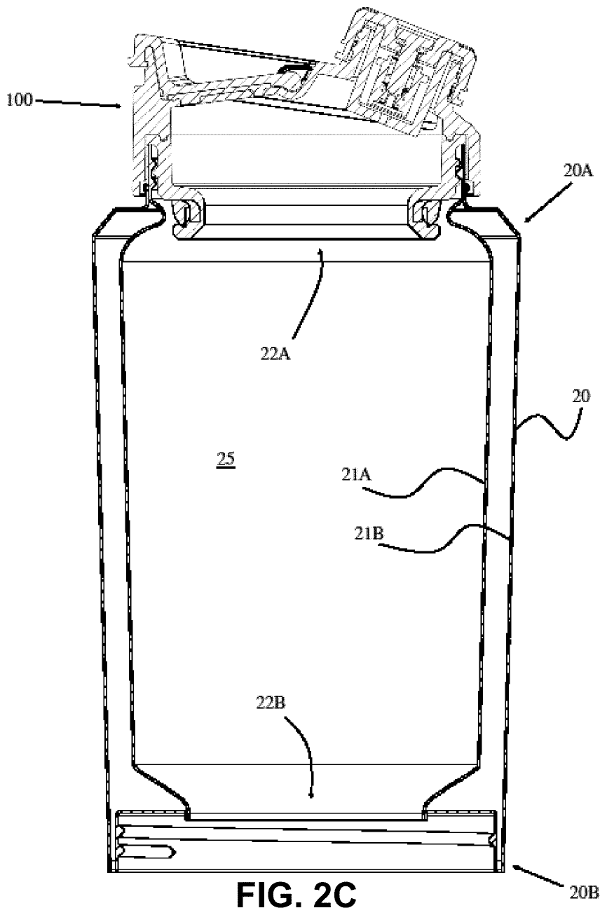

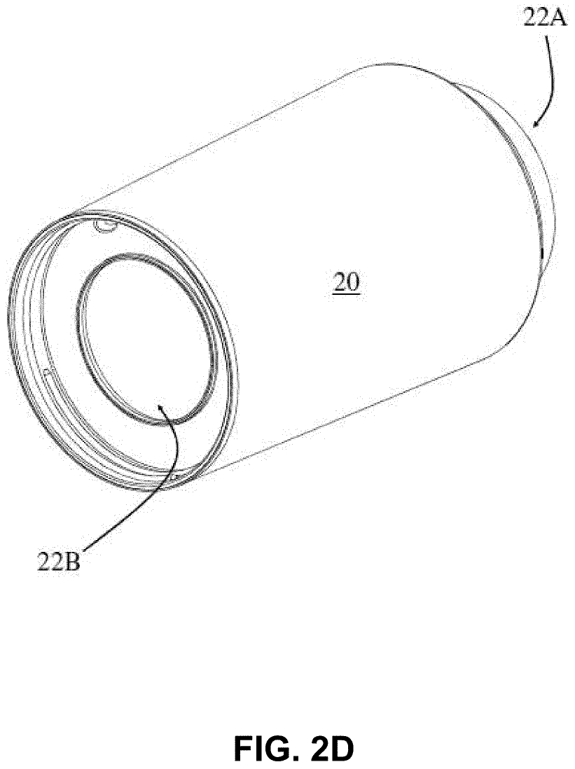

Other embodiments may also include storage module (not illustrated) which can provide storage space, for example, within an at least partially enclosed pocket or other like compartment. In this manner, a user may store keys, money, a wallet, food, tea and/or other items as desired. Moreover, the main body portion 20 of the container 10 is shown in FIGS. 2A, 2B, 2C and 2D. For instance, as shown in the cut-away view of FIG. 2C, the body 20 of at least one embodiment includes a double wall construction comprising an inner wall 21A and an outer wall 21B. The inner wall 21A and outer wall 21B are spaced apart from one another to define an area or gap there between, at least along a portion of the body 20. In at least one embodiment, the inner wall 21A and outer wall 21B are sealed to one another proximate the top end 20A and bottom end 20B of the body 20 in order to seal the area or gap there between.

In some embodiments, the area between the inner wall 21A and outer wall 21B is vacuum sealed and/or includes an insulating material in order to reduce or restrict the dissipation of heat from the fluid or contents within the container 20 and through the body 20 or wall(s) 21A, 21B thereof.

In at least one embodiment, both the inner wall 21A and the outer wall 21B are constructed of a metallic or Stainless Steel material, and in other embodiments or implementations, the inner wall 21A may be constructed of a metal or metallic material, whereas the outer wall 21B may be constructed of a plastic material. However, it should be noted that other materials for the inner and outer walls, as well as other portions and components of the container 10 are contemplated within the full spirit and scope of the present invention.

Furthermore, the body 20 of at least one embodiment includes an at least partially open top 22A through which the fluid (or other contents of the container) can be filled or dispensed. A lid 100 can be secured or removable connected to the open top 22A of the body 20, as illustrated, for example, and configured for allowing consumption of the contents directly from the container 10. Additionally, as shown in FIGS. 2C and 2D, in at least one embodiment, the body 20 includes an at least partially open bottom 22B. As described herein, the heating assembly 30 of at least one embodiment is attached (e.g., either removably or fixedly) to the body 20 of the container 10. In this manner, the heating assembly 30, and in particular, the heating element thereof, may be directly exposed to the interior portion 25 of the container 10 (e.g., where the fluid or other contents are contained), and therefore, the heating assembly 30 or heating/cooling element thereof may be disposed in direct contact with the fluid or other contents of the container 10, for instance, through the at least partially open bottom end 22B. The direct contact between the contents of the container 10, such as water, fluid, etc., further facilitates the container 10 to quickly, effectively and efficiently increase the temperature of the fluid or other contents to a desired temperate or temperature range.

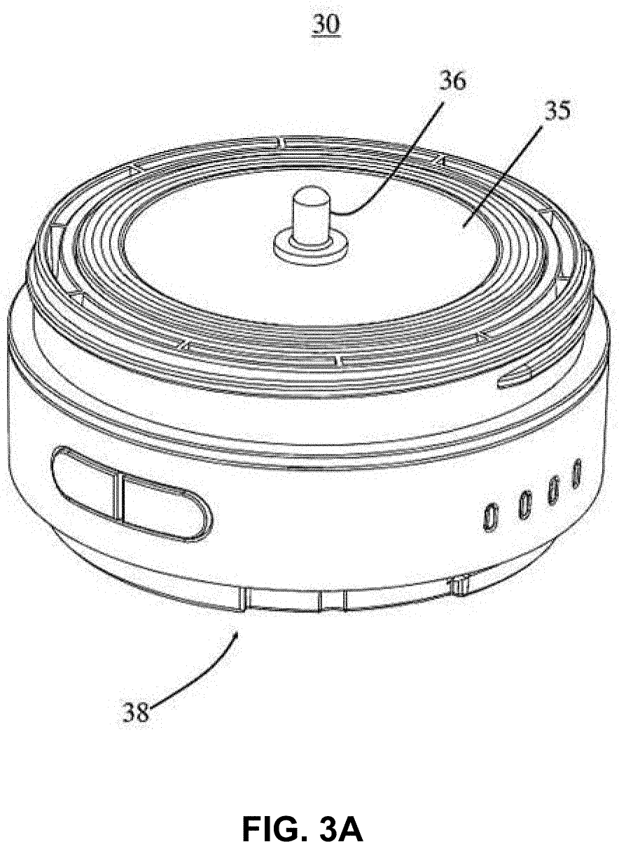

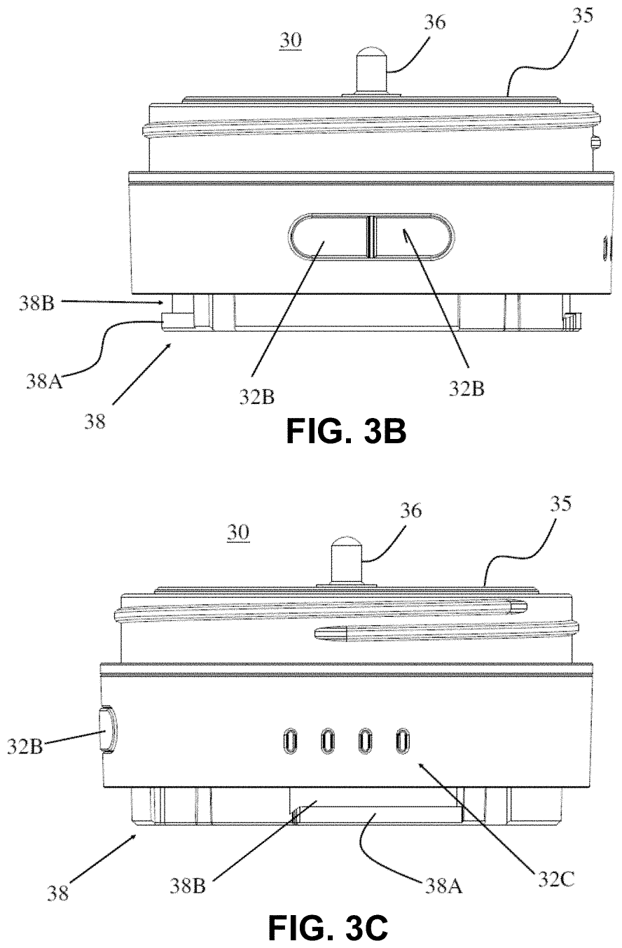

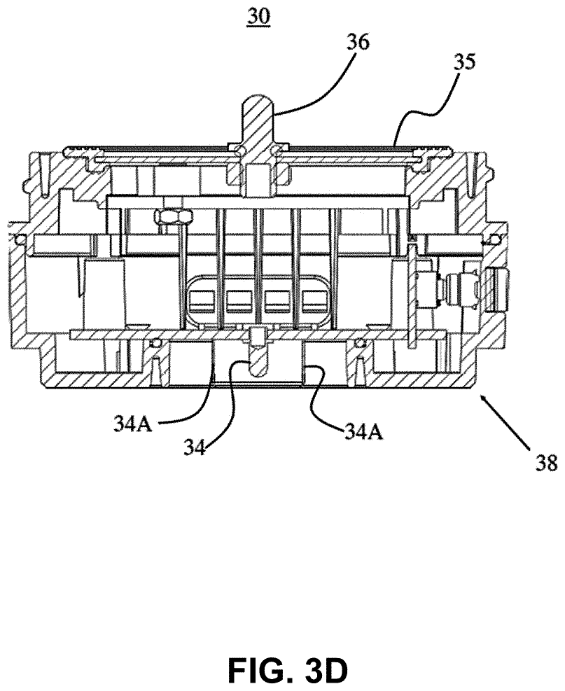

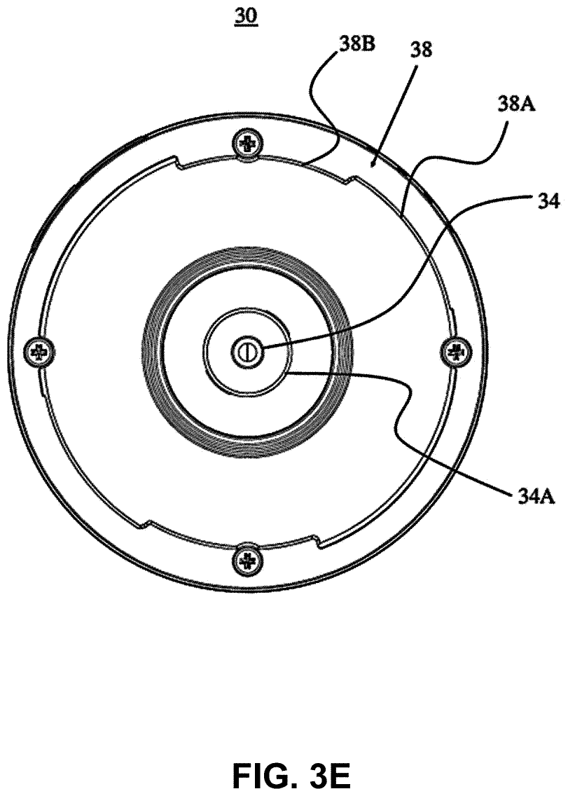

The at least partially open bottom 22B and/or direct and physical contact between the contents of the container 10 and the heating assembly 30 or heating element 35 thereof also facilitates the container 10 in heating the fluid or other contents to or above a boiling point temperature, which, in the case of water is 212 degrees Fahrenheit. As an example, at least a portion of the heating element 35, such as at least a portion of the heating panel(s), etc., is exposed and in direct physical contact with the contents of the container 10, such as the water or other fluid, liquid, etc. This facilitates a fast and efficient heating system that can heat the contents to high temperate, up to and including a boiling point, such as 212° F. Moreover, with reference now to FIGS. 3A through 3F, a heating assembly 30 of at least one embodiment is illustrated. In particular, the heating assembly 30 includes an at least partially exposed heating element 35 that is adapted to increase in temperature upon application of electricity or power, for example, from a power source, including a battery pack or electrical cable.

As shown, the heating element 35 is exposed on the top of the heating assembly 30, such that, when the heating assembly 30 is attached or secured to the main body portion 20 of the container 10, the heating element 30 is aligned with or otherwise disposed at least partially within the open bottom 22B. In this manner, any contents, including water, fluid, beverage, soup, etc., disposed within the body 20 of the container 10, will be in direct contact with the heating element 35.

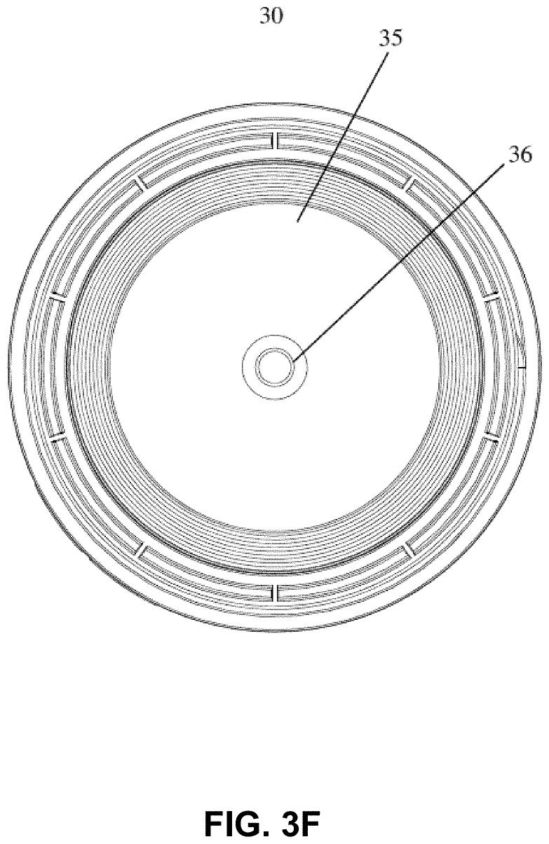

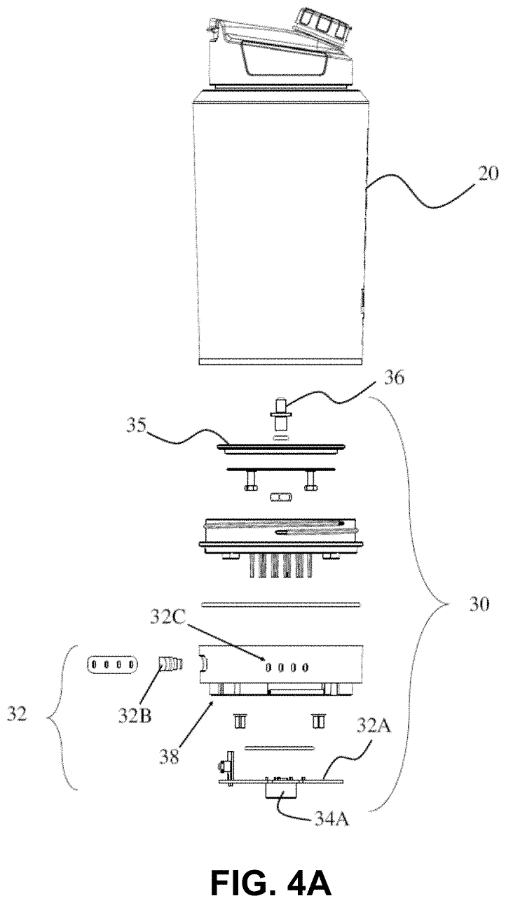

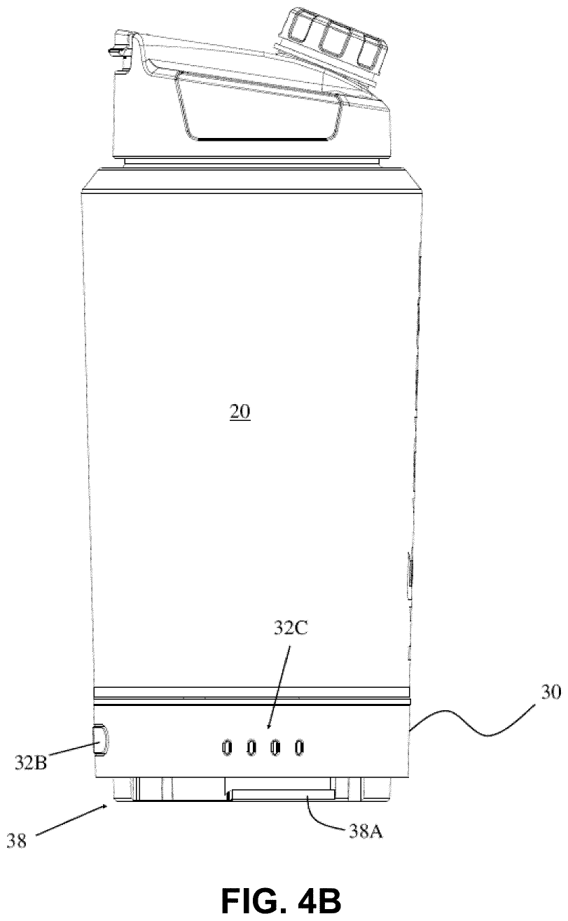

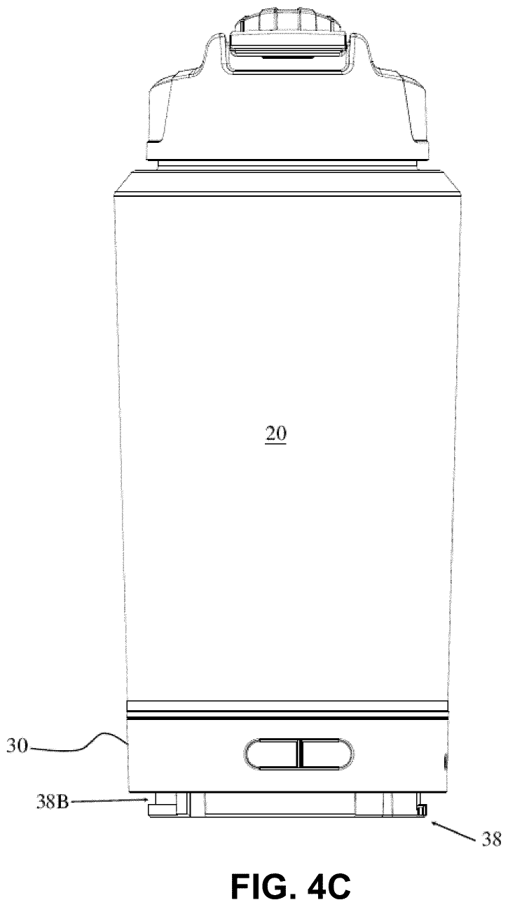

Furthermore, with reference to FIG. 4A, for example, the heating assembly 30 of at least one embodiment may also include a control assembly, generally referenced as 32, structured to control the heating and/or cooling of the heating elements 35, for example, by supplying or eliminating power or electricity thereto. For instance, the control assembly 32 can include one or more circuit boards, generally referenced as 32A, and/or one or more control buttons or switches 32B structured to select a temperature mode, temperature setting, and/or power the heating assembly on or off, as an example.

Some embodiments can include one or more status LEDs or lights, as generally referenced at 32 C, in order to provide a visual representation of the temperature, setting, mode, etc. of the heating assembly 30. As an example, in at least one embodiment, the heating assembly 30, and in particular the control assembly 32 thereof, may include a plurality of heating modes which can be selected by the user, for example, by selecting one or more of the control buttons or switches 32B, for example. In one implementation, the heating assembly 30 includes two or more, for example, three, heating or ‘maintain’ modes, each of which represent a different preset, predefined or user preselected temperature or temperature range. For example, in at least one embodiment, the different temperature ranges or modes may include: (a) 124° F.-134° F., (b) 135° F.-145° F., (c) 160° F.-170° F., and (d) 194° F.-204° F. In some embodiments, the different temperatures may include, for example: (a) 104° F., (b) 140° F., (c) 176° F., and (d) boil or 212° F.

For instance, a user may select one of the temperatures or temperature ranges by pressing one or more of the control buttons 32B on the heating assembly 30, e.g., either a short tap or a long press. In operation, when the temperature of the contents of the container 10 falls below the preset, predefined or user preselected temperature, the heating element 35 will be activated until the temperature of the contents is raised to the preset, predefined or user preselected temperature. This cycle will continue so long as the heating assembly 30 remains activated and in a selected temperature mode. As provided above, in at least one embodiment, the heating assembly 30 can include a ‘boil’ mode in which the temperature of the contents is raised to a boiling temperature (e.g. 212° F.). This is accomplished at least in part because of the direct contact between the heating element 35 and the contents of the container 10, as well as the amount of power and electricity that is supplied thereto.

Furthermore, and with reference still to FIGS. 3A through 3D and 3F, at least one embodiment of the present invention further includes a temperature sensor 36 connected, attached or exposed on the top end of the heating assembly 30. As shown, the temperature sensor 36 may be in the form of a node or probe that extends at least partially from the top of the heating assembly 30. In some embodiments, the heating element(s) 35 may be disposed at least partially around an inner circumferential portion of the heating assembly 30 with the temperature sensor 36 extending upward from the center thereof, although other configurations are certainly contemplated within the full spirit and scope of the present invention.

Furthermore, it should be noted that the temperature sensor 36 or probe may extend at least partially into the main body of the container 10 in a manner such that it comes into direct physical contact with the contents of the container 10, such as a heated liquid. This allows the temperature sensor 36 or probe to obtain accurate and/or precise temperature readings of the contents. For instance, as described herein, when the temperature of the contents is reduced or falls below a certain selected temperature range, the heating element 30 may be activated to raise the temperature. When the temperature sensor 36 or probe detects the temperature of the contents as being within the selected range or approximately at the selected temperate, then the heating elements 30 of some embodiments may turn off or be reduced in temperature. This cycle will continue at least while a temperature or temperature range is selected or activated on the container 10 or heating assembly 30.

FIGS. 4B through 4D illustrate the heating assembly secured to the bottom end of the main body portion 20 of the container 10. In some embodiments, the heating assembly 30 may be fixedly attached or connected to the main body portion 20 in that it may not intended to be easily or readily removed. In other embodiments, the heating assembly 30 can be removably attached to the main body portion 20, for example, via cooperative threaded components or other attachment assemblies that provide a liquid tight seal. In such an embodiment, removing the heating assembly 30 can expose the open bottom 22B of the main body portion 20, which can provide access to the interior of the container 10 thereby. This can help with the cleaning of the interior portion of the container 10.

Furthermore, with reference to FIGS. 3E, 4A, and 4D, the heating assembly 30 includes one or more electrical connections, such as or prongs 34 and ring 34A, configured to electrically connect with a power source module 40, 50, 60 such as a battery pack module or AC/DC power module, for example. Particularly, the electrical connections 34, 34A of at least one embodiment are structured to electrically connect with a separate power source module 40, 50, 60 as described herein, and are capable of transferring power or electricity from the power source module 40, 50, 60 to the heating assembly 30 or the heating element 35, thereof, for example, as controlled by the control assembly 32. In the embodiment shown, the electrical connections of the heating assembly 30 may include a prong or probe 34 that extends downward and an outer ring 34A that at least partially or completely encircles or surrounds the prong 34. The prong 34 and ring 34A may both be constructed of a metal or metallic material configured to facilitate an electrical connection.

One of the connections, such as prong 34 may be a positive terminal, while the other connection 34A may be a negative terminal, although the polarity of the terminals 34, 34A may be reversed. In any event, as described herein, the prong 34 will be engaged by a corresponding peg, pegs or other electrical connection(s) of a corresponding module 40, 50, 60 and the ring 34A will be engaged by another peg, pegs or other electrical connection(s). This design or configuration allows the additional module, e.g., a power source module 40, 50, 60 to electrically connect to the heating assembly 30, while also allowing the heating assembly 30 and/or module 40, 50 60 to twist or rotate relative to one another without the electrical connections being dislodged or losing contact. Furthermore, a connection assembly 38 is also included in at least one embodiment of the heating assembly 30 of the present invention. The connection assembly 38 is structured to facilitate selective interconnection between the heating assembly 30 and the power source or additional module 40.

As illustrated, the connection assembly 38 of at least one embodiment may include a twist and lock mechanism such that the bottom end of the heating assembly 30 can cooperatively interconnect with the top end of the additional or power source module 40, 50, 60 by engaging the heating assembly 30 and the additional or power source module 40, 50, 60 and twisting the heating assembly 30 (or the connected main body portion 20), for example, a half or quarter revolution. In this manner, the connection assembly 38 of at least one exemplary embodiment may include one or more ledges 38A, grooves 38B, etc. that are structured to cooperatively interconnect with similarly shaped corresponding ledges or grooves on the top end or top portion of the additional or power source module.

Accordingly, twisting, locking or otherwise connecting the heating assembly 30 to the additional or power source module 40, 50, 60 allows the main body 20, the heating assembly 30, and the additional or power source module 40, 50, 60 to be raised, lowered and/or otherwise transported as a single connected unit. Other connection assemblies, including, snaps, hooks, recesses, grooves, etc. can be used in accordance with the various embodiments of the present invention. It should also be noted that, in at least one embodiment, the main body portion 20 and the heating assembly 30 can be easily disconnected from the additional or power source module(s) 40, 50, 60. In this regard, a user can keep the power source module 40, 50, 60 sitting on a table, in a cup holder, etc. while the main body portion 20 and the heating assembly 30 can be raised and lowered as a unit for drinking purposes. Each time the user sets the main body portion 20 and the heating assembly 30 down, he or she can set it upon the power source module 40, 50, 60 for continued heating and/or operation thereby.

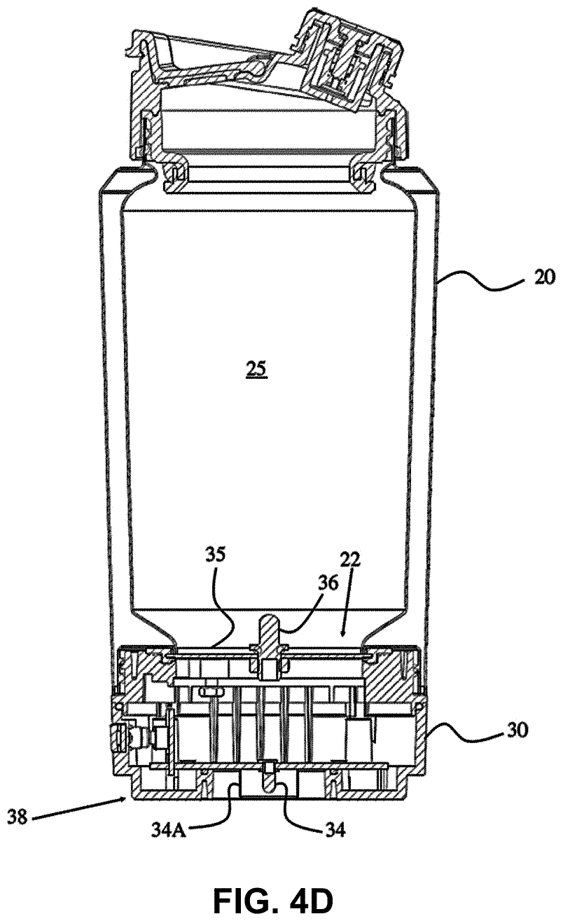

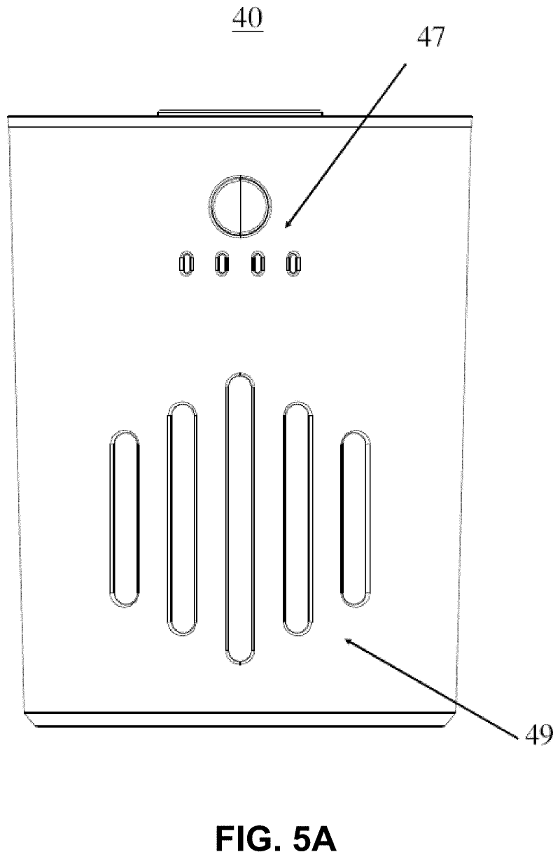

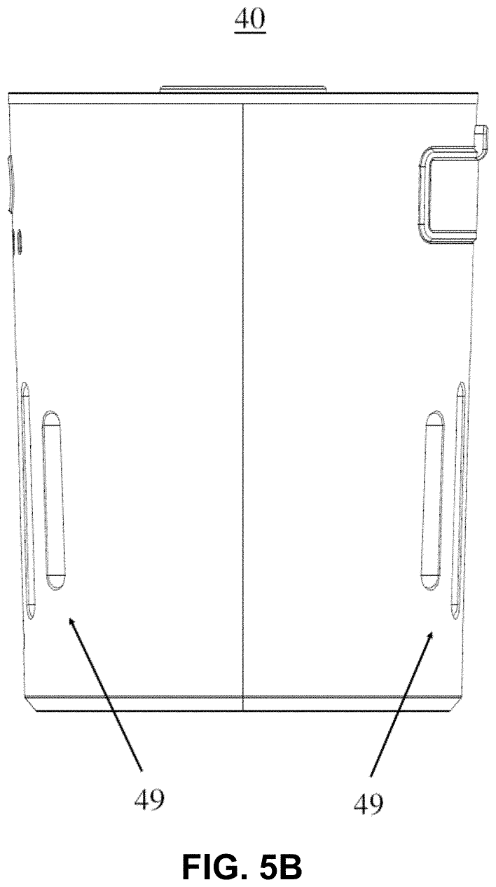

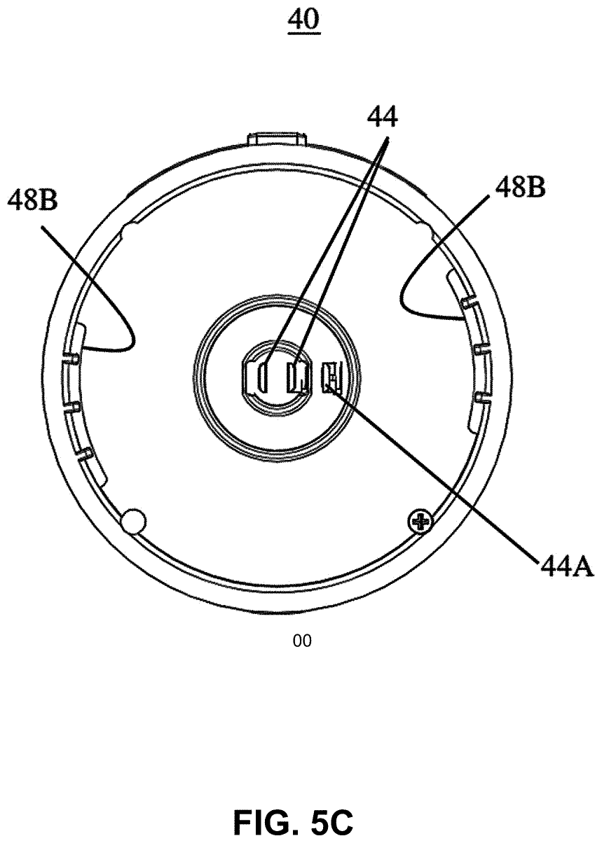

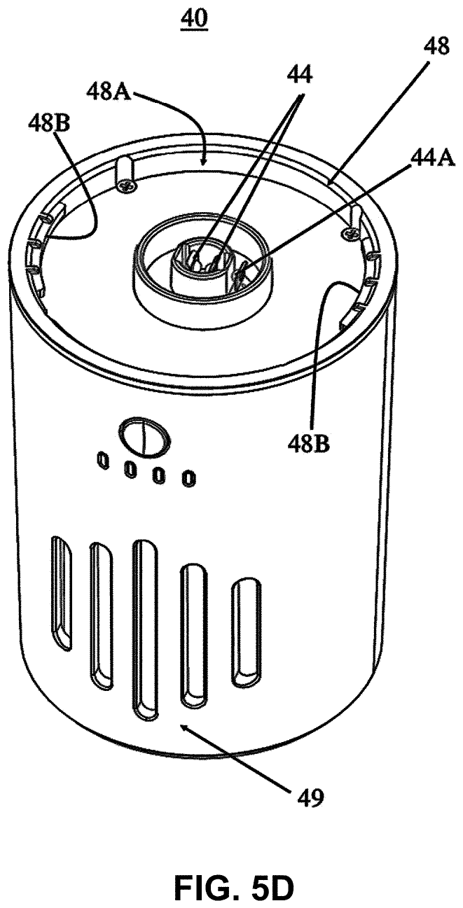

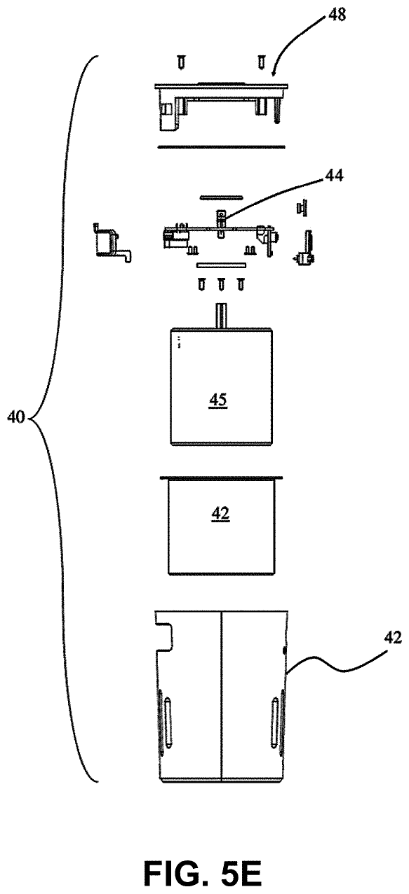

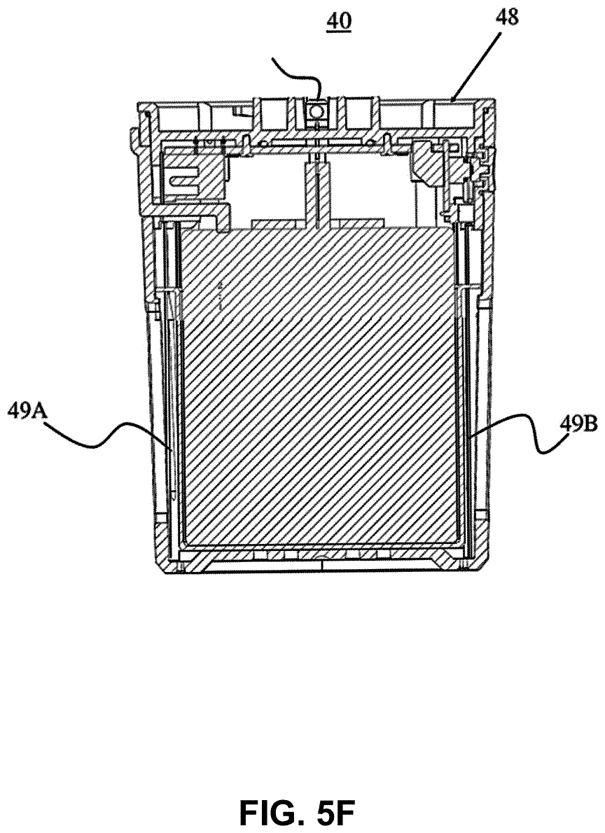

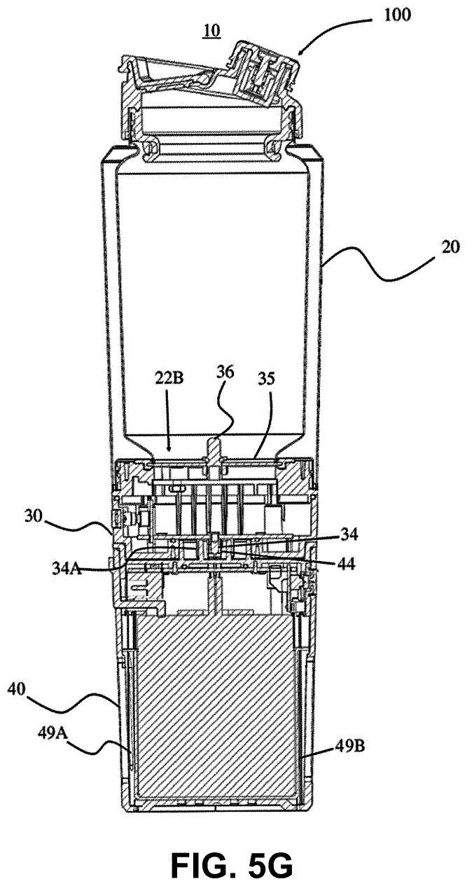

Accordingly, in some embodiments, the heating assembly 30 may be electrically connected to the additional or power source module without engaging the connection assemblies or otherwise without locking the heating assembly 30 to the additional or power source module. Other embodiments may require the connections assemblies to be locked or engaged in order to ensure or provide an electrical connection there between. With reference now to FIGS. 5A through 5F, one embodiment of the additional or attachment module 40, such as a power source module, is illustrated. In this embodiment, the module 40 includes a battery pack in that it incorporates at least one, and in most cases, a plurality of batteries, such as rechargeable batteries. In such a case, the module 40 can be plugged into an external power source, such as an AC or DC power outlet to recharge the batteries. In any event, with reference to FIGS. 5A though 5D, the module 40 includes a connection end 48 which is structured to interconnect with the connection assembly 38 of the heating assembly 30. For instance, as shown in FIG. 5D, the connection end 48 of the module 40 may include one or more recesses 48A with one or more peripheral flanges 48B. With the connection assembly 38 of the heating assembly 30 disposed at least partially within the recess 48A, the one or more flanges 48B will engage or connect with the heating assembly 30, for example, upon twisting the connection assembly 38 relative to the module 40.

For instance, in some embodiments, the one or more flanges 48 may slide within one or more grooves 38B and/or engage one or protrusions or ledges 38A of the connection assembly 38. As provided above, other connection assemblies can be implemented within the full spirit and scope of the present invention. Furthermore, as shown in the top view of FIG. 5C, the module 40 includes at least one electrical connection 44, 44A configured to receive or otherwise electrically connect with the electrical connection 34, 34A of the heating assembly 30. In the embodiment illustrated, the connection 44 of the module 40 includes at least two prongs 44 or two electrical connections within which the electrical connection 34 or prong(s) of the heating assembly 30 can be disposed. An outer peg or electrical connection 44A of at least one embodiment is structured to engage or contact ring 34A. Upon doing so, the electrical connections 34, 34A and 44, 44A of the heating assembly 30 and module 40, respectively, will electrically interconnect with one another thereby allowing the power or electricity from the module 40 to flow or transfer to the heating assembly 30.

Also, this design allows the heating assembly 30 and/or module 40 to spin, twist or rotate (for example partially or 360 degrees) relative to one another without the electrical contacts being disconnected. This is accomplished via the ring 34A and the outer contact 44A. With reference now to the exploded view of FIG. 5E, the module 40 of at least one embodiment includes a plurality of batteries 45, disposed within a housing 42 and electrically connected to connection 44. In at least one embodiment, the module 40 may include at least four (4) batteries, and in some embodiments eight (8) or more batteries. The module 40 and batteries 45 thereof, provide high current capable of providing enough electricity to the heating assembly 30 for the heating assembly 30 to boil water and/or otherwise operate in the intended manner, as described herein.

In one exemplary embodiment, the module 40 may include eight (8) lithium rechargeable batteries (e.g., ICR18650 batteries) with approximately 2600 mAh each, although other batteries with different capabilities and specifications are contemplated within the various embodiments of the present invention. Moreover, in one embodiment, four (4) batteries may be connected in series, with at least two (2) connected in parallel. In one exemplary embodiment, the beginning current may be approximately 15 A, with a working current between 11 A and 14 A.

One or more LEDs or status lights 47 may be included and visible external to the module 40 in order to visually reference or determined the current battery level or electrical charge remaining on the battery pack or module 40. It should also be noted that certain embodiments of the present invention may also include a cooling assembly structured to facilitate the effective dissipation of heat that may be generated by the battery pack or module 40. For instance, the cooling assembly of at least one embodiment may include one or a plurality of ventilation holes, generally referenced as 49, disposed on at least some portions of the housing. The ventilation holes may be arranged on one or more sides and/or bottom surfaces of the housing.

Furthermore, it should be noted that it takes a tremendous amount of energy to boil water, which can create a significant amount of heat, for example, in the battery module 40 or other modules 50, 60, described herein. Accordingly, additional components or features of the cooling assembly, which may be implemented in the battery module 30 or other modules disclosed herein, may also include one or more heat sinks, generally referenced as 49A and 49B, disposed at least partially within or otherwise connected to the module 40, 50 or 60. For instance, with reference to FIGS. 5F and 5G, in some cases, the heat sink(s) 49A, 49B, may be disposed along one or more side surfaces of the module, such as along or proximate one or more ventilation holes 49, in order to facilitate in the dissipation of heat and to prevent potential overheating of the module or otherwise in order to prevent or minimize the module being overly hot to the touch. The heat sink(s) 49A, 49B of certain embodiments may include one more sheets or panels, whether flat, corrugated or otherwise, of metal configured to dissipate the heat.

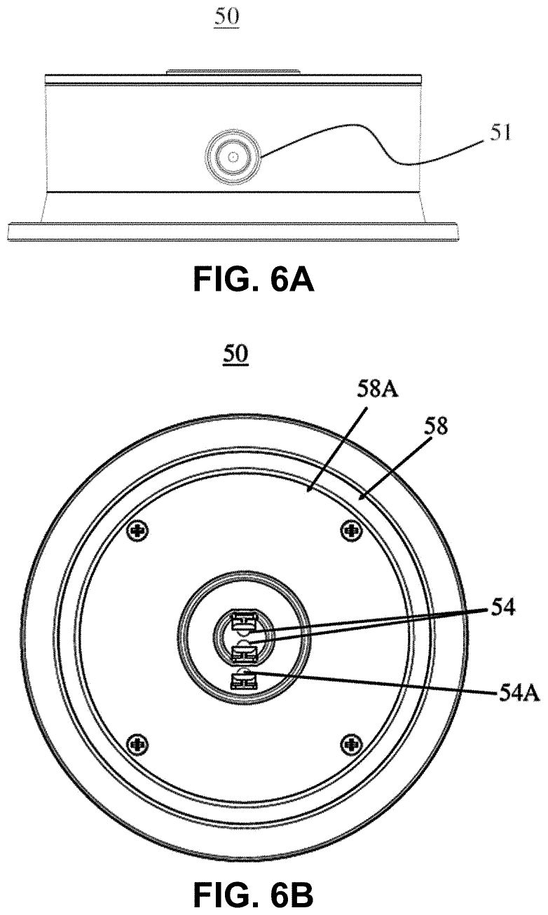

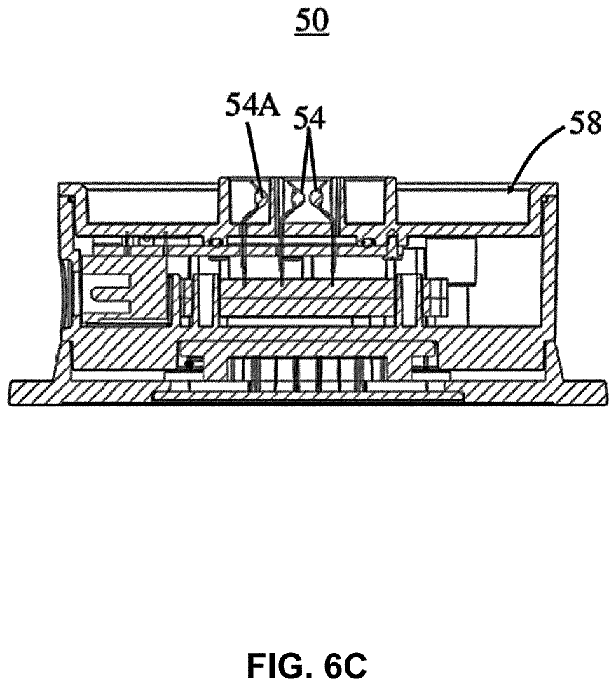

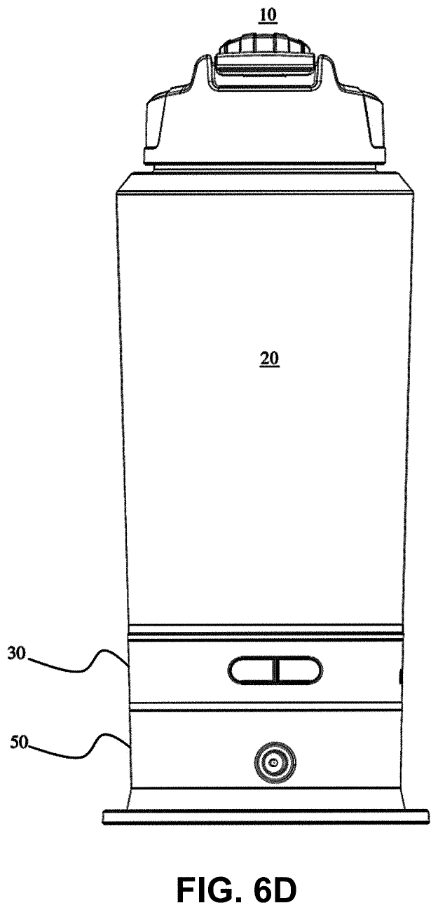

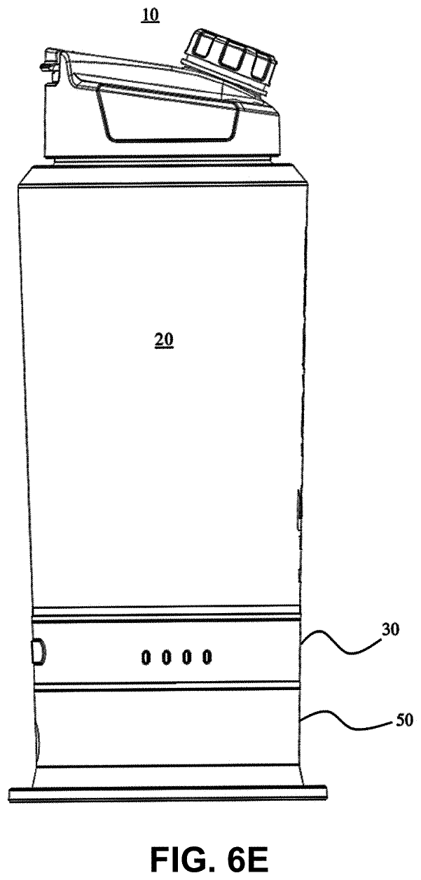

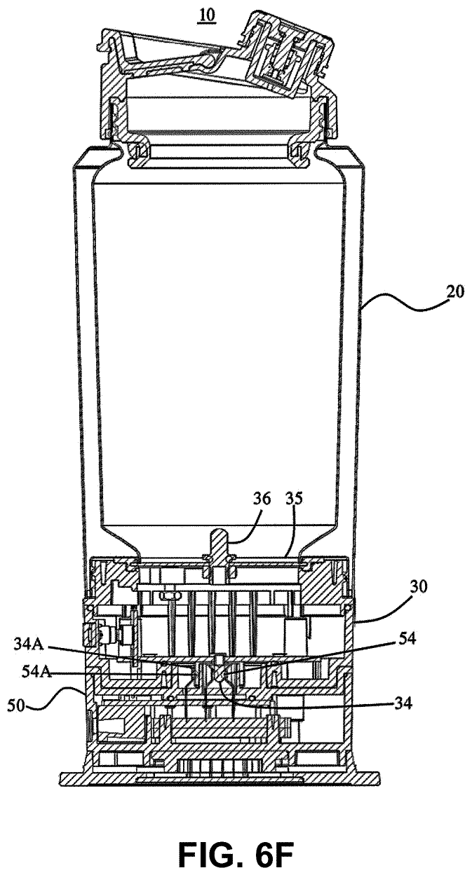

Other embodiments may also include one or more heat sinks disposed along the bottom surface of the module (not shown). FIGS. 6A through 6F illustrate another embodiment of an additional or power source module 50. In this embodiment, a connection 51 is provided for connecting the module 50 to an external power source, such as an AC electrical outlet, for example, via an electrical or power cable (not shown). The external power source can then provide the power though the corresponding cable to the module 50, which in turn is electrically connected to the heating assembly 30. For instance, the module 50 illustrated in FIGS. 6A through 6F can be selectively connected or disconnected with the heating assembly 30 in the same manner as the module 40 described above and illustrated in FIGS. 5A through 5F.

For example, the module 50 of at least one embodiment includes a connection end 58 which is structured to interconnect with or otherwise at least partially receive the connection assembly 38 of the heating assembly 30. For instance, connection end 58 of the module 50 may include recesses 58A within which the connection assembly 38 or a portion of the heating assembly 30 may sit or reside. In at least one embodiment, as illustrated in FIG. 6B, the recess 58A of the module 50 may not include any flanges (such as flanges 48B of the battery module 40). In such a case, the heating assembly 30 and/or container 10 is able to be easily lifted out of the module 50 such that the module 50 may remain seated on a support surface such as a desk top or counter top. This allows a user to lift the container off of the module 50, for example, in order to drink out of the container or pour contents from the container, and subsequently seat the container back upon the module 50 in order to resume heating.

Accordingly, in some embodiments, the container, heating assembly 30 and/or module 50 may include a memory chip or memory capabilities such that the container and/or heating assembly 30 is lifted off of the module 50, and then subsequently return to the module 50 such that an electrical connection is reestablished between the heating assembly 30 and the module 50, the previous settings (e.g., the previously selected temperature range) does not need to be re-entered by the user.

Other embodiments may include one or more flanges (not shown in FIG. 6B) such that the module 50 may lock within the heating assembly 30, in a similar manner as the battery module 40 discussed herein. Furthermore, as shown in the top view of FIG. 6B, the module 60 includes at least one electrical connection 54, 54A configured to receive or otherwise electrically connect with the electrical connection 34, 34A of the heating assembly 30. In the embodiment illustrated, the connections 54 of the module 50 include at least two prongs or electrical connections within which the electrical connection 34 or prong(s) of the heating assembly 30 can be disposed. An outer peg or electrical connection 54A of at least one embodiment is structured to engage or contact ring 34A. Upon doing so, the electrical connections 34, 54 of the heating assembly 30 and module 50, respectively, will electrically interconnect with one another thereby allowing the power or electricity from the module 50 to flow or transfer to the heating assembly 30. Also, as described above with regard to the module 40, this design allows the heating assembly 30 and/or module 50 to spin, twist or rotate (for example partially or 360 degrees) relative to one another without the electrical contacts being disconnected.

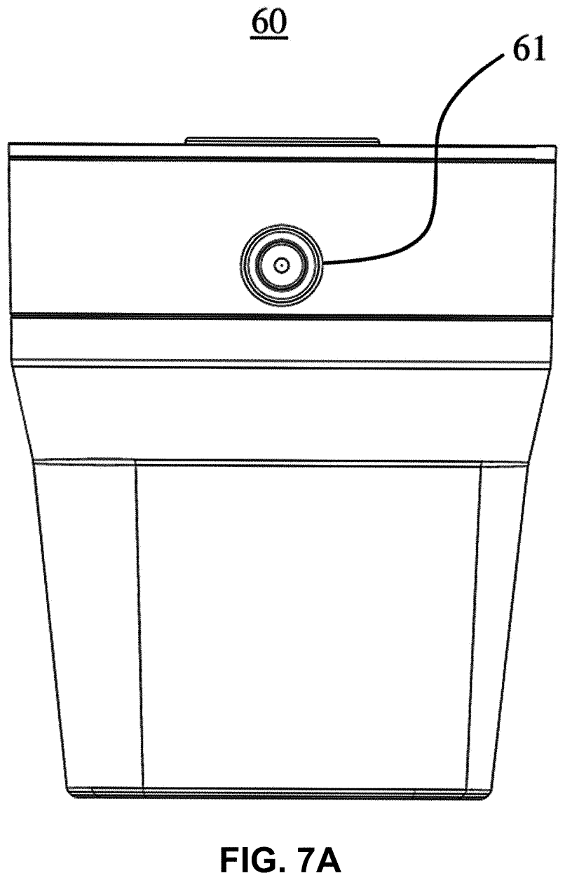

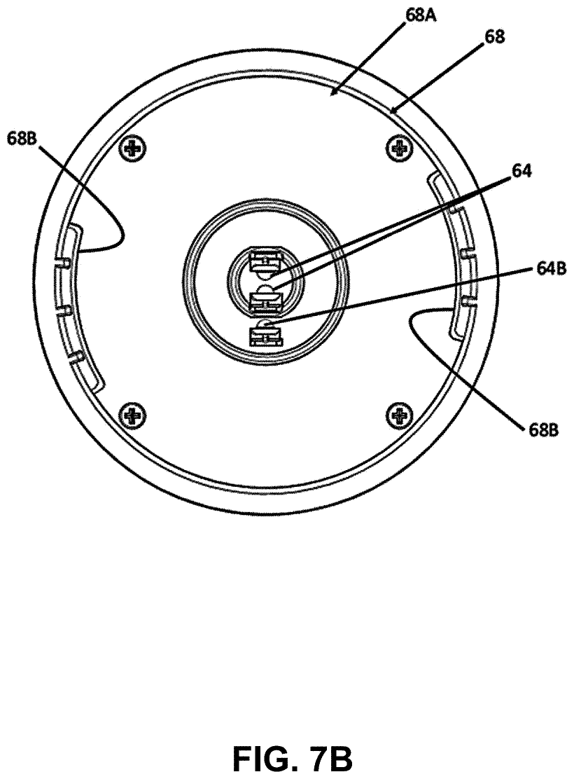

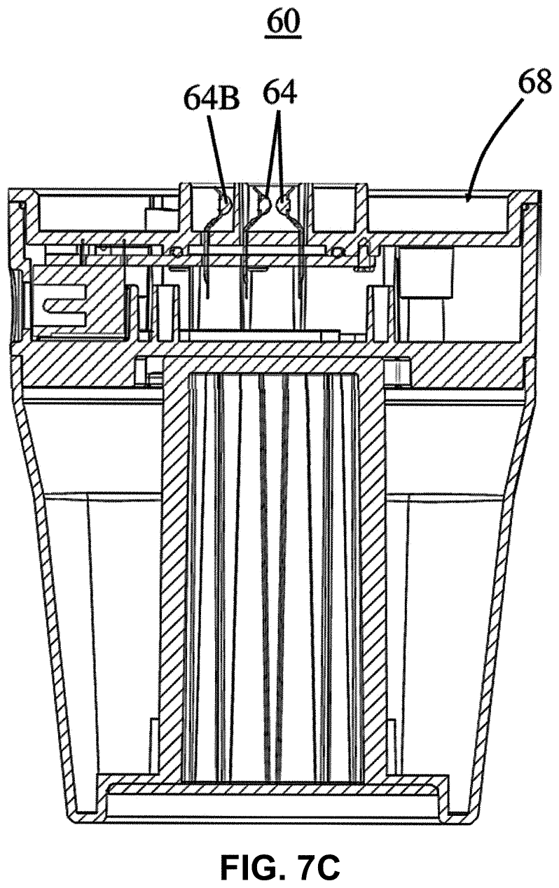

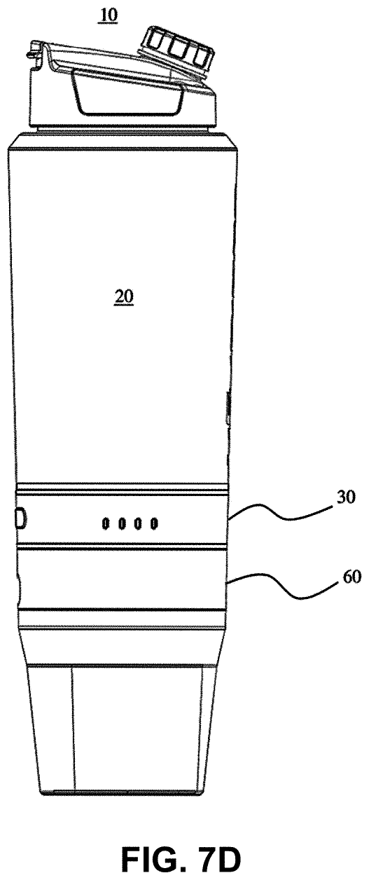

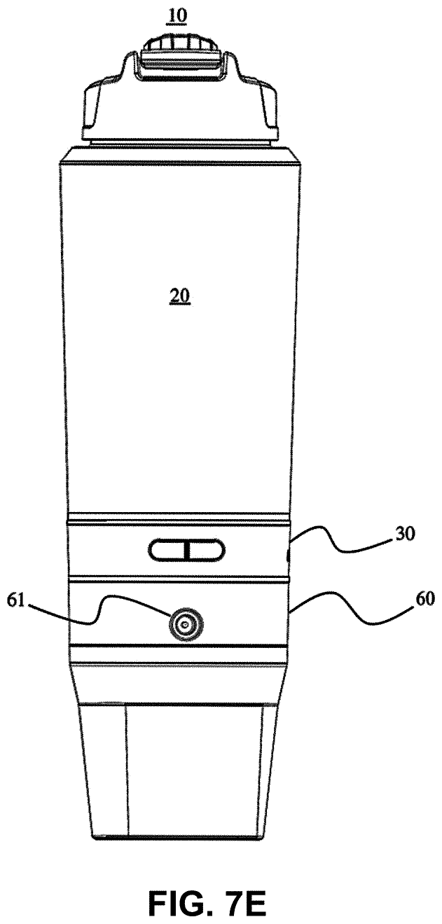

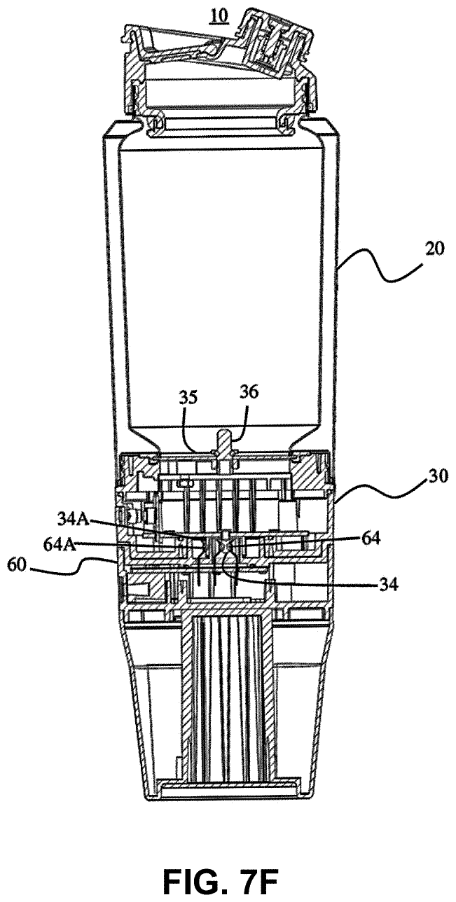

FIGS. 7A through 7F illustrate yet another embodiment of an additional or power source module 60. In this embodiment, a connection 61 is provided for connecting the module 50 to an external power source, such as a vehicle DC electrical outlet, for example, via an electrical or power cable (not shown). The external power source can then provide the power though a corresponding cable to the module 60, which in turn is electrically connected to the heating assembly 30. For instance, the module 60 illustrated in FIGS. 7A through 7F can be selectively connected or disconnected with the heating assembly 30 in the same manner as the module 40 described above and illustrated in FIGS. 5A through 5F. For example, the module 60 of at least one embodiment includes a connection end 68 which is structured to interconnect with the connection assembly 38 of the heating assembly 30. For instance, connection end 68 of the module 60 may include recesses 68A with one or more peripheral flanges 68B. With the connection assembly 38 of the heating assembly 30 disposed at least partially within the recess 68A, the one or more flanges 68B will engage or connect with the heating assembly 30, for example, upon twisting the connection assembly 38 relative to the module 50.

Furthermore, as shown in the top view of FIG. 7B, the module 60 includes at least one electrical connection 64, 64A configured to receive or otherwise electrically connect with the electrical connection 34, 34A of the heating assembly 30. In the embodiment illustrated, the connection 64 of the module 60 includes at least two prongs within which the electrical connection 34 or prong(s) of the heating assembly 30 can be disposed. An outer peg or electrical connection 64A of at least one embodiment is structured to engage or contact ring 34A. Upon doing so, the electrical connections 34, 64 of the heating assembly 30 and module 60, respectively, will electrically interconnect with one another thereby allowing the power or electricity from the module 60 to flow or transfer to the heating assembly 30. Also, this design allows the heating assembly 30 and/or module 60 to spin, twist or rotate (for example partially or 360 degrees) relative to one another without the electrical contacts being disconnected.

In addition, the module 60 illustrated in FIG. 7A though 7F includes a specifically configured base or housing that can fit within a cup holder, for example, those commonly found in the cabin of a car or other vehicle. In use, the module 50 may sit within the cup holder while the cable is connected to the vehicle's power source, such as through the DC power supply cable.

If desired, the user can selectively disconnect the main body portion 20 and the heating assembly 30 from the module 60, for example, via the twist and lock (or other) connection assembly. This can allow the user to drink from the container 10 while the module 60 remains seated within the cup holder or upon a table, counter, etc. Setting the heating assembly 30 back upon the module 60 will reconnect the electrical connections 34, 64, resuming heating operations.

Accordingly, in some embodiments, the heating assembly 30, and the connection assembly 38 thereof, need not be locked into place with the module 40, 50, 60 for the module 40, 50, 60 to operate and/or otherwise to deliver power or electricity from the module 40, 50, 60 to the heating assembly 30.

Furthermore, in some embodiments, the container 10 includes a memory component in order to store the selected settings or modes. For example, when the main body portion 20 is removed from the module 40, 50 60 and then returned to the module 40, 50 60, when it is returned to the module 40, 50, 60 and reconnected, the heating assembly 30 will remember the prior selected settings or modes (e.g., temperature range(s)) and continue to heat the contents of the container 10 according to those settings or modes.

### A Collapsible Pump Assembly for Water Purification Applications at Outdoor Remote Locations:

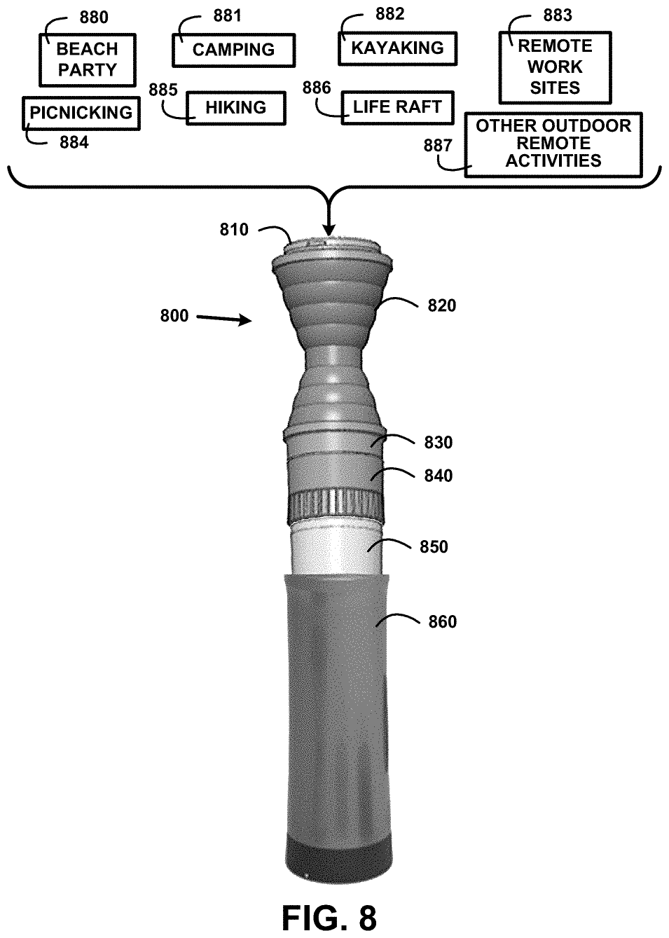

FIG. 8 shows for illustrative purposes only an example of a collapsible pump assembly for water purification applications at outdoor remote locations of one embodiment. FIG. 8 shows a collapsible pump assembly for water purification applications at a beach party 880, camping 881, kayaking 882, remote work sites 883, picnicking 884, hiking 885, life raft 886, and other outdoor remote activities 887. The collapsible pump assembly for water purification is also referred to herein with the terms “water purification unit”, “modular liquid purification assembly” and “attachable water purification apparatus” without any change in meaning.

An illustrative example is a camping 881 trip where a campsite could be setup anywhere including in a forest, next to a lake, or on the beach of an ocean, where a potable safe water supply is not available. In one embodiment the water purification unit 800 includes a closure system 840 is a manually operated valve. The valve is closed when pouring water into the collapsible pump assembly 820 to hold the impure water in the collapsible pump assembly 820 chamber. The UV cap 810 ultraviolet light radiations is initiated and when the disinfection process is completed the closure system 840 valve is manually opened to allow the now disinfected water to flow to a filter box module 850 for filtering out particulates and microbial organisms. The filter box module 850 includes at least one carbon or plant based filter element and biocide element. The purified water flows into a container bottle 860 ready for use in drinking, for use in cooking, brewing coffee and other purified water uses.

In yet another embodiment the water purification unit 800 is provided with at least one digital processor, at least one digital memory device, at least one digital biological detector and analyzer, at least one digital chemical detector and analyzer, at least one digital controller, and at least one digital valve. The user can pour a source of impure water including tap water or dirty water into the top of the container. The water purification unit 800 on top includes an ultraviolet (UV) LED. The UV cap 810 provides ultraviolet light to disinfect the water poured through the top into the collapsible pump assembly 820.

The at least one digital biological detector and analyzer use sensors to detect the presence of viruses, bacteria and microorganisms in the water. When the at least one digital biological detector and analyzer does not detect the presence of viruses, bacteria and microorganisms in the water a closure system 840 coupled to a connector 830 at the bottom of the collapsible pump assembly 820 opens a valve allowing the water to flow through a filter box module 850. The filter box module 850 removes particulates and chemicals in the water as the water flows into the container bottle 860. The disinfected and filtered water is potable regardless of its source. The purified water can be used for drinking, to make coffee, cooking and for making other beverages of one embodiment.

### Container Water Purification Modules:

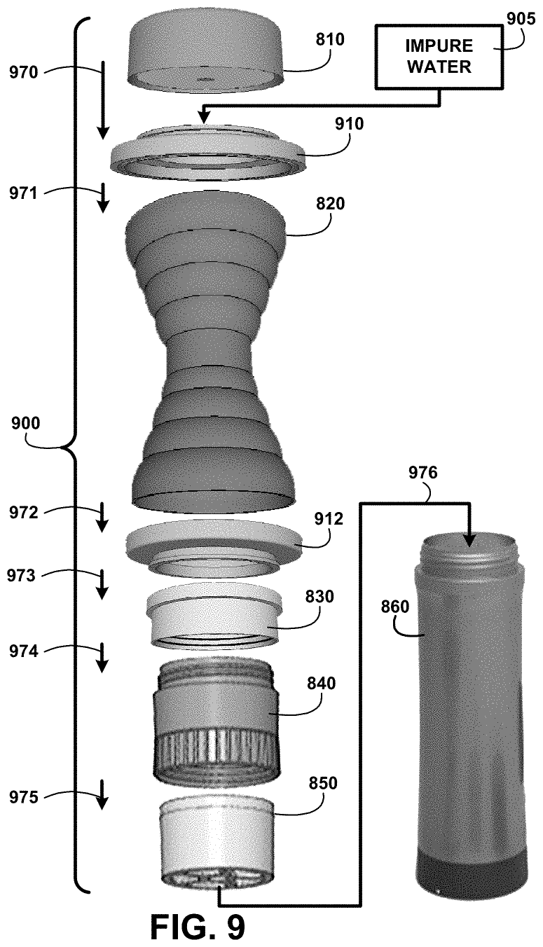

FIG. 9 shows for illustrative purposes only an example of water purification modules of one embodiment. FIG. 9 shows portable water purification unit modules 900. The portable water purification unit modules 900 when assembled provide a means to purify impure water 905. Impure water 905 is poured into the collapsible pump assembly 820. A connector ring 910 couples to the collapsible pump assembly 820 to permit other modules to be coupled to the collapsible pump assembly 820. In this example a first connector ring 910 is coupled to the top of the collapsible pump assembly 820 971. The UV cap 810 is coupled to the first connector ring 910 970. The UV cap 810 is used to radiate ultraviolet light into the impure water 905 poured into the collapsible pump assembly 820. The collapsible pump assembly 820 is coupled to a second connector ring 912 972. The second connector ring 912 is coupled to the collapsible pump assembly connector 830 973. The collapsible pump assembly connector 830 provides a screw coupling for other modules. The close system 840 is coupled to the collapsible pump assembly connector 830 974. The close system 840 provides a valve that remains closed until the UV cap 810 radiations has completed exposing microorganisms to UV radiation disrupting virus, bacteria and microorganism DNA and disables an ability to replicate thereby disinfecting the water. The close system 840 is coupled to the filter box module 850 975. When the disinfection process is complete the close system 840 valve is opened allowing the disinfected water to flow through the filter box module 850 975.

The filter box module 850 includes at least one filter mesh to prevent particulates and microbial organisms from flowing through to a container. The filter box module 850 is ultrasonically sealed to prevent any leakage. The filter box module 850 includes at least one carbon or plant based filter element and biocide element. The filter box module 850 is coupled to the container bottle 860 which receives the purified water 976 of one embodiment.

### UV Cap Module:

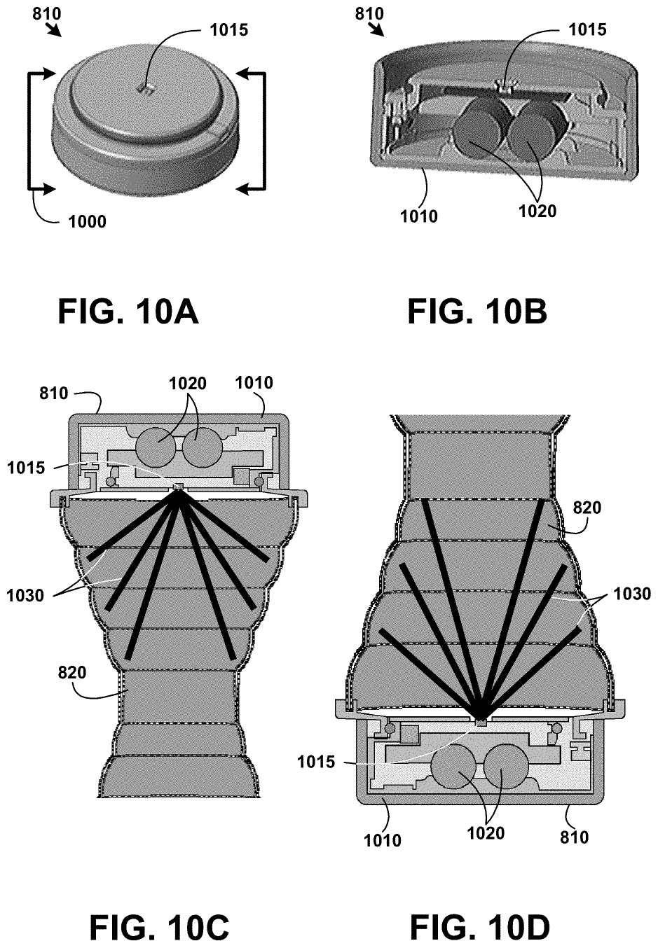

FIG. 10A shows for illustrative purposes only an example of a UV cap module of one embodiment. FIG. 10A shows the UV cap module 810. The UV cap module 810 includes an ultraviolet LED light 1015. Also showing are cross section indicators 1000 for the cross section seen in FIG. 10B of one embodiment.

### UV Cap Module Cross Section:

FIG. 10B shows for illustrative purposes only an example of a UV cap module cross section of one embodiment. FIG. 10B shows a cross section of the UV cap module 810. The cross section interior view shows the batteries 1020 that provide power to the ultraviolet LED light 1015. Also showing is a connection housing 1010 used for coupling the UV cap module 810 to other modules of one embodiment.

### UV Cap Module Radiating Ultraviolet Light in the Collapsible Pump Assembly Top Section:

FIG. 10C shows for illustrative purposes only an example of a UV cap module radiating ultraviolet light disinfecting the collapsible pump assembly top section of one embodiment. FIG. 10C shows the UV cap module 810 radiating ultraviolet light 1030 disinfecting the collapsible pump assembly 820 top section. The UV cap module 810 is coupled to the top of the collapsible pump assembly 820 using the connection housing 1010. The batteries 1020 are providing power to the ultraviolet LED light 1015 providing radiating ultraviolet light 1030 to disinfect the empty collapsible pump assembly 820 top section prior to the next purification use of one embodiment.

### UV Cap Module Radiating Ultraviolet Light Disinfecting the Collapsible Pump Assembly Bottom Section:

FIG. 10D shows for illustrative purposes only an example of a UV cap module radiating ultraviolet light disinfecting the collapsible pump assembly bottom section of one embodiment. FIG. 10D shows the UV cap module 810 radiating ultraviolet light 1030 disinfecting the collapsible pump assembly 820 bottom sections. The UV cap module 810 is coupled to the bottom of the collapsible pump assembly 820 using the connection housing 1010. The batteries 1020 are providing power to the ultraviolet LED light 1015 providing radiating ultraviolet light 1030 to disinfect the empty collapsible pump assembly 820 bottom section prior to the next purification use of one embodiment.

### Filter Box Module:

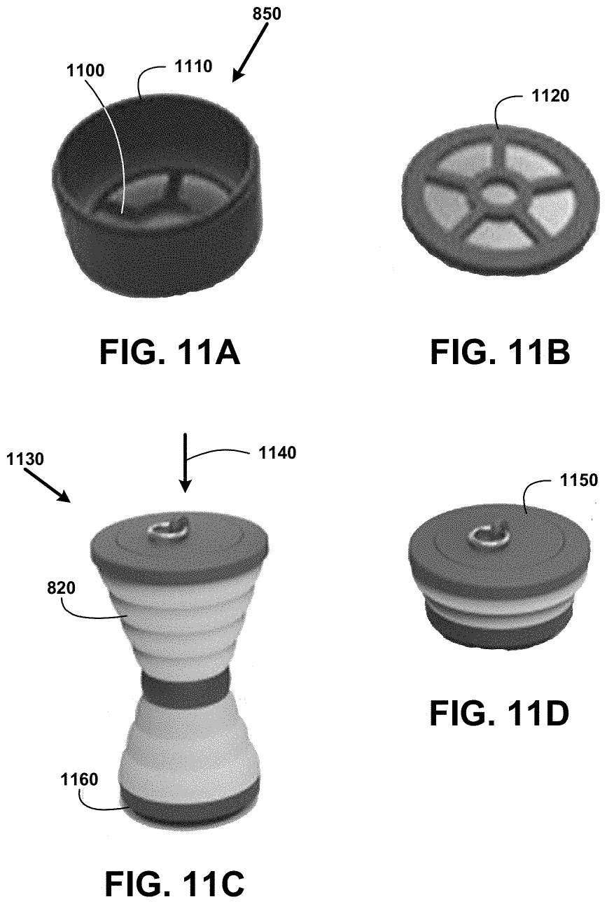

FIG. 11A shows for illustrative purposes only an example of a filter box module of one embodiment. FIG. 11A shows the filter box module 850 module including an interior view of a filter box housing 1110. The filter box housing 1110 will hold multiple filter mesh component elements 1100. In one embodiment the filter box module 850 module includes a carbon or plant based filter element added filtration for improved taste and odor reduction. The filter mesh component elements 1100 include a primary filter biocidal element for filtering out microbial organisms of one embodiment.

### Filter Mesh Element:

FIG. 11B shows for illustrative purposes only an example of a filter mesh element of one embodiment. FIG. 11B shows a filter mesh element 1120. The filter mesh element 1120 includes a mesh supporting frame and a mesh screen. In a plurality of filter mesh elements the size of the mesh screens is varied to filter out specific particulates and microbial organisms of one embodiment.

### Collapsible Pump Assembly Extended:

FIG. 11C shows for illustrative purposes only an example of a collapsible pump assembly extended of one embodiment. FIG. 11C shows the collapsible pump assembly 820 in an extended condition 1130. The collapsible pump assembly 820 is collapsible when pushed 1140 to collapse in one embodiment.

### Collapsible Pump Assembly Compressed:

FIG. 11D shows for illustrative purposes only an example of a collapsible pump assembly collapsed of one embodiment. FIG. 11D shows the collapsible pump assembly 820 in a collapsed condition 1150. The collapsible pump assembly 820 includes an accordion structure that compresses upon itself when not in use for storage. The compressed condition makes the collapsible pump assembly 820 much smaller for carrying in for example a backpack and other storage units used for example on a camping or hiking trip. The collapsible pump assembly 820 when pulled from both ends expands as shown in FIG. 11C of one embodiment.

### A First Crossing Double Electrode Module:

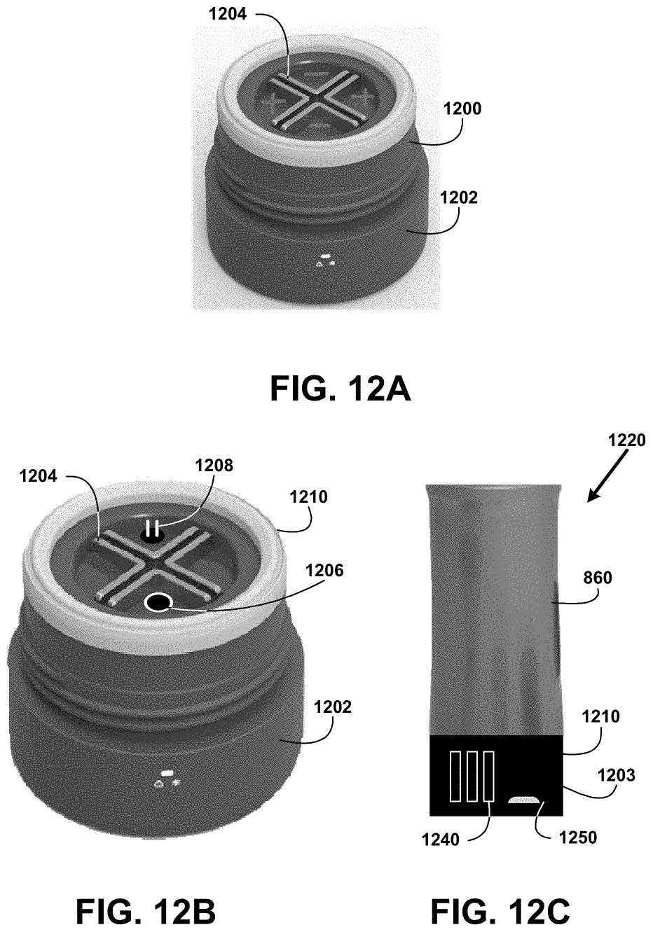

FIG. 12A shows for illustrative purposes only an example of a first crossing double electrode module of one embodiment. FIG. 12A shows a first crossing double electrode 1200 is attachable to the container bottle 860 of FIG. 8. The crossing double electrodes 1204 are powered by a battery pack 1202. The first crossing double electrode 1200 performs an electrolysis process to produce purified hypochlorous acid (HOCL) an effective non-toxic disinfectant of one embodiment.

### A Second Crossing Double Electrode Module:

FIG. 12B shows for illustrative purposes only an example of a second crossing double electrode module of one embodiment. FIG. 12B shows a second crossing double electrode module 1210 is provided with a UV LED 1206 for radiating the liquid HOCL with ultraviolet light for disinfecting water to ensure only pure water is used for electrolysis. The second crossing double electrode module 1208 is provided with a pH sensor 1208 for detecting the pH level of the purified HOCL solution. The crossing double electrodes 1204, UV LED 1206 and pH sensor 1208 are powered by a battery pack 1202. The second crossing double electrode 1210 performs an electrolysis process to produce purified hypochlorous acid (HOCL) an effective non-toxic disinfectant. The electrolysis produced HOCL will effectively maintain the UV LED 1206 disinfected purity of the liquid by attacking and disabling any viruses, bacteria and microorganisms preventing any regrowth of one embodiment.

### Portable HOCL Generator Assembly:

FIG. 12C shows for illustrative purposes only an example of a portable HOCL generator assembly of one embodiment. FIG. 12C shows a portable HOCL generator assembly 1220 for generating HOCL from purified water and a pure salt using electrolysis for processing the purified water and the pure salt. The portable HOCL generator assembly 1220 is configured with attachable modules including the liquid container bottle 860, second crossing double electrode 1210, batteries 1240, and USB port 1250. The USB port can be connected to an external power source using a USB connection. The electrical power conducted to the first crossing double electrode 1200 and second crossing double electrode 1210 includes in one embodiment a phase pulsed electrical charge circuit and device 1203 creating faster and more precise electrolysis process of one embodiment.

In another embodiment the HOCL generator assembly for generating HOCL from purified water and a pure salt using electrolysis for processing the purified water and the pure salt is made with a larger diameter for greater volume. The larger diameter HOCL generator assembly includes the second crossing double electrode 1210 components, the phase pulsed electrical charge circuit and device 1203 components, and the batteries 1240, and USB port 1250.

In yet another embodiment HOCL generator assembly for generating HOCL from purified water and a pure salt using electrolysis for processing the purified water and the pure salt is made with stackable couplings on the top and bottom. The stackable couplings may include electrical couplings allowing power to be conducted from one stacked HOCL generator assembly to another stacked HOCL generator assembly. The stackable couplings may include fluid couplings with open/close valves allowing liquid to flow from one stacked HOCL generator assembly to another stacked HOCL generator assembly.

Multiple HOCL generator assembly units may be grouped to increase the volume of HOCL at a single location of one embodiment.

### Filtering Impure Water:

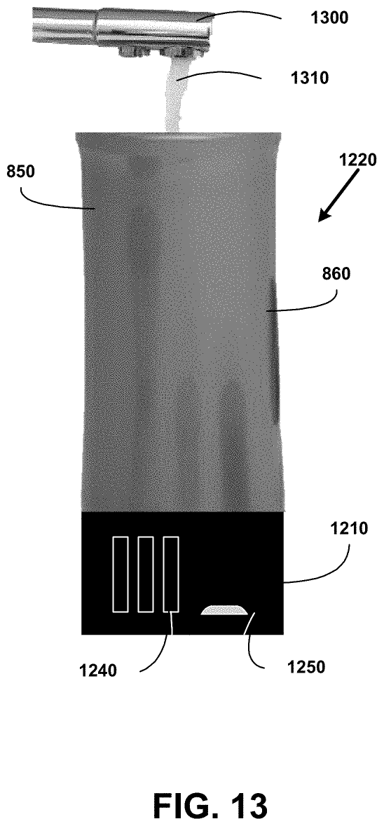

FIG. 13 shows for illustrative purposes only an example of filtering impure water for generating HOCL of one embodiment. FIG. 13 shows a portable HOCL generator assembly 1220. The uncapped portable HOCL generator assembly 1220 is filled with impure water for example tap water 1310 from a well faucet 1300. The impure water passes through the filter box module 850 inside the container bottle 860 and not shown. The filter box module 850 holds multiple filter mesh component elements 1100 of FIG. 11A. In one embodiment the filter box module 850 module includes a carbon or plant based filter element added filtration for improved taste and odor reduction. The filter mesh component elements 1100 of FIG. 11A include a primary filter biocidal element for filtering out microbial organisms. The filtered purified water fills the container bottle 860 in preparation for electrolysis processing of one embodiment.

Depositing a Pure Salt into the Filtered Water:

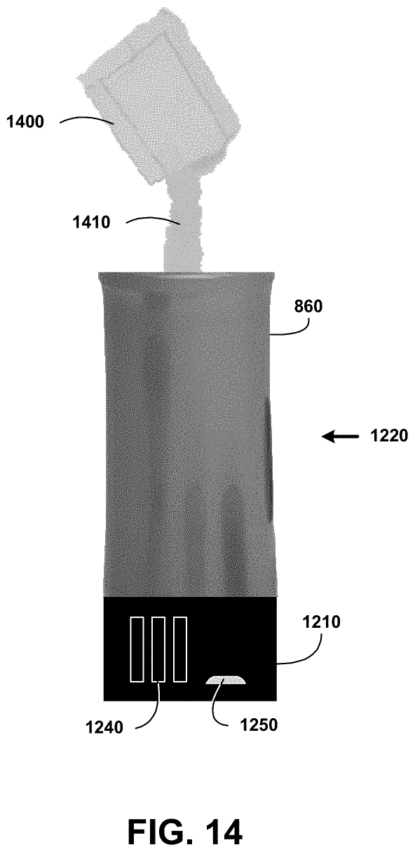

FIG. 14 shows for illustrative purposes only an example of depositing a pure salt into the filtered water of one embodiment. FIG. 14 shows the portable HOCL generator assembly 1220. A packet 1400 with a predetermined amount of the pure salt 1410 is deposited into the filtered water in the container bottle 860 with the second crossing double electrode 1210, batteries 1240, and USB port 1250 of one embodiment.

### An Electrolysis Process:

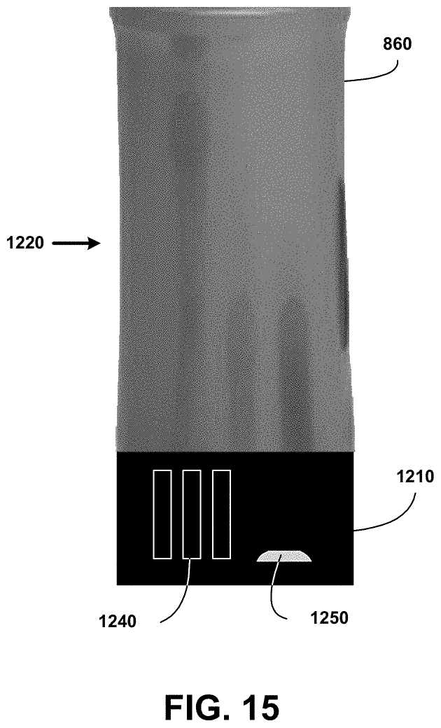

FIG. 15 shows for illustrative purposes only an example of an electrolysis process of one embodiment. FIG. 15 shows the second crossing double electrode 1210, batteries 1240, and USB port 1250 coupled to the container bottle 860. Power from the batteries 1240 is transmitted to illuminate the UV LED 1206 of FIG. 12B for radiating the filtered water to expose any microorganisms to UV radiation disrupting virus, bacteria and microorganism DNA and disables an ability to replicate thereby disinfecting and purifying the water.

Power from the batteries 1240 is transmitted to the second crossing double electrodes 1204 of FIG. 12B to begin an electrolysis process. The electrolysis process creates an electrical current in the purified water and pure salt mixture. The electrical current triggers a non-spontaneous chemical reaction. Electrolysis of the pure salt and purified water generates hypochlorous acid (HOCL).

The pH sensor 1208 of FIG. 12B integrated into the second crossing double electrode 1210 detects the pH level of the HOCL solution and transmits this data to a portable HOCL generator app of one embodiment.

### Portable HOCL Generator Sprayer:

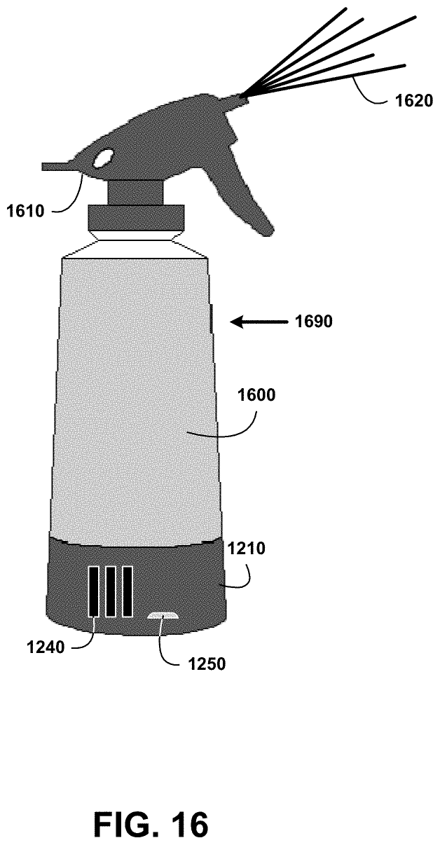

FIG. 16 shows for illustrative purposes only an example of a portable HOCL generator sprayer of one embodiment. FIG. 16 shows a portable HOCL generator sprayer 1690. The portable HOCL generator sprayer 1690 includes a sprayer bottle 1600 with the second crossing double electrode 1210. A sprayer pump 1610 is coupled at the top of the sprayer bottle 1600. The portable HOCL generator sprayer 1690 allows a user to spray 1620 HOCL on to surfaces and objects for disinfecting the surface or object. The second crossing double electrode 1210 with the batteries 1240, and USB port 1250 provides includes the pH sensor 1208 of FIG. 12B. The pH sensor 1208 of FIG. 12B integrated into the second crossing double electrode 1210 detects the pH level of the HOCL solution and transmits this data to a portable HOCL generator app of one embodiment.

### Portable HOCL Generator App:

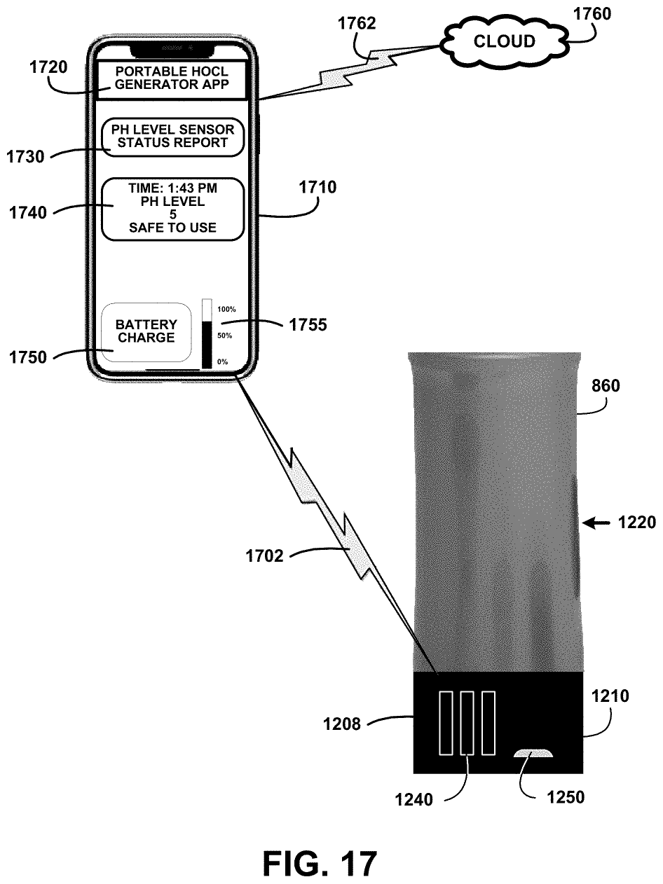

FIG. 17 shows for illustrative purposes only an example of a portable HOCL generator app of one embodiment. FIG. 17 shows the portable HOCL generator assembly 1220 coupled with the second crossing double electrode 1210, batteries 1240, and USB port 1250 coupled to the container bottle 860.

The second crossing double electrode 1210 pH sensor 1208 is transmitting 1702 with a communication device for example a cellular device, near-field communication device, a Bluetooth device or a WI-FI device to a user digital device 1710. The user digital device 1710 includes a portable HOCL generator app 1720 for receiving and sending information to at least the portable HOCL generator assembly 1220.

The pH sensor 1208 transmits data showing the pH level sensor status report 1730. The pH sensor 1208 transmitted data includes for example the time: 1:43 pm, pH level 5, safe to use 1740. This data communication keeps the user informed of the pH level status reflect the status of the HOCL. Too high a pH level over 7 indicates the HOCL may not be effective and a too low pH level below 3 indicates the HOCL is acidic to a point of being dangerous and may have become chlorine.

The portable HOCL generator assembly 1220 also transmits to the portable HOCL generator app 1720 a battery charge 1750 reading for example 65% on a percentage scale 1755. The portable HOCL generator app 1720 also transmits 1762 the portable HOCL generator assembly 1220 data to a cloud 1760. The user may review and store the portable HOCL generator assembly 1220 data transmitted to the cloud 1760 for records of HOCL production and location where the HOCL was applied of one embodiment.

### Filter Box Housing:

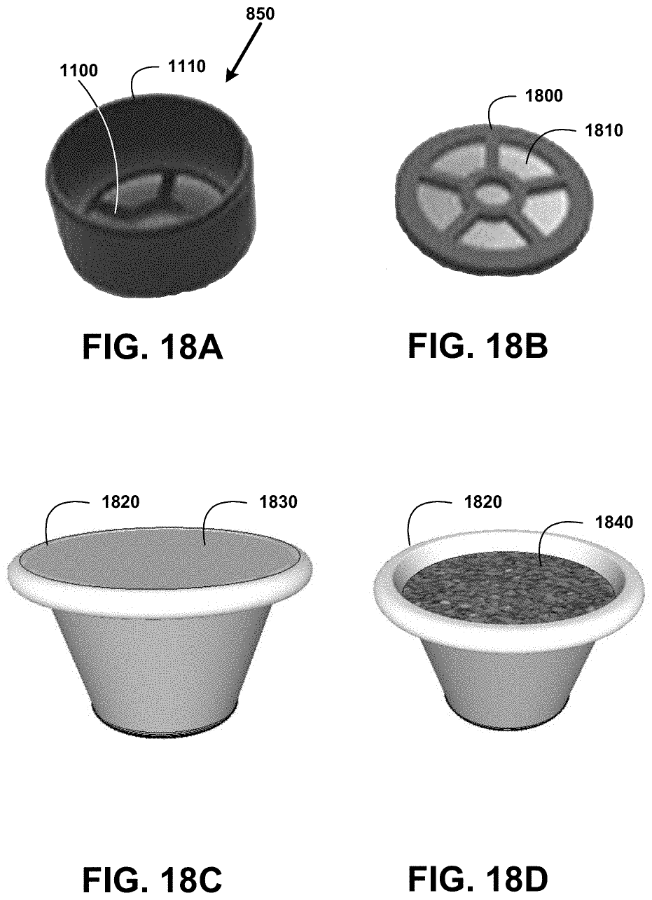

FIG. 18A shows for illustrative purposes only an example of a filter box housing of one embodiment. FIG. 18A shows the filter box module 850 for placing filter mesh component elements 1100 in the filter box housing 1110. A user may select different filter mesh component elements 1100 for different purposes for example a coffee filter for brewing coffee and an asymmetric membrane filter for desalination of salt water of one embodiment.

### Coffee Filter:

FIG. 18B shows for illustrative purposes only an example of a coffee filter of one embodiment. FIG. 18B shows a coffee filter 1800 configured with a stainless steel mesh 1810. A stainless steel mesh 1810 does not need replacing. Paper filters remove key acids, using the stainless steel mesh 1810 the brewed coffee will have more robust flavor of one embodiment.

### Preloaded Cup:

FIG. 18C shows for illustrative purposes only an example of preloaded cup of one embodiment. FIG. 18C shows a preloaded cup 1820 containing coffee grounds for loading into the filter box housing 1110 of FIG. 11A. The preloaded cup 1820 includes a preloaded cup sealed cover 1830 to keep the coffee ground inside fresh. A user may also elect to put one or more coffee grounds of their choice into the filter box housing 1110 of FIG. 11A of one embodiment.

### Coffee Ground Blend:

FIG. 18D shows for illustrative purposes only an example of a coffee ground blend of one embodiment. FIG. 18D shows the preloaded cup 1820 with coffee ground blend 1840 grounds within the cup. A user may select a single coffee ground bean choice or a blend of multiple coffee bean choice grounds to brew of one embodiment.

### Collapsible Pump Assembly Coffee Brewing:

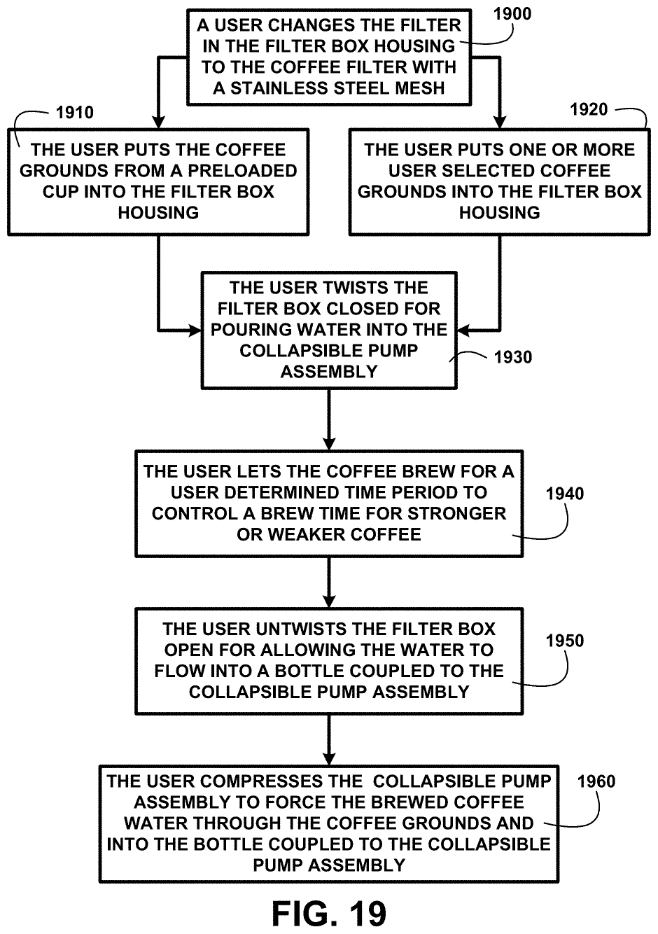

FIG. 19 shows a block diagram of an overview of collapsible pump assembly coffee brewing of one embodiment. FIG. 19 shows a user's collapsible pump assembly coffee brewing steps. A user changes the filter in the filter box housing to the coffee filter with a stainless steel mesh 1900. The user puts the coffee grounds from a preloaded cup into the filter box housing 1910. In another embodiment the user puts one or more user selected coffee grounds into the filter box housing 1920 for brewing.

The user twists the filter box closed for pouring water into the collapsible pump assembly 1930. The user lets the coffee brew for a user determined time period to control a brew time for stronger or weaker coffee 1940. The user untwists the filter box open for allowing the water to flow into a bottle coupled to the collapsible pump assembly 1950. The user compresses the collapsible pump assembly to force the brewed coffee water through the coffee grounds and into the bottle coupled to the collapsible pump assembly 1960. The collapsible pump assembly coffee brewing is happening in a sealed assembly that will not leak allowing the user to be brewing coffee while carrying the collapsible pump assembly in their hand, a backpack or other transportable means. The coffee brewing will continue for as long as the user determines.

Once the user has allowed the brewing to take place for the strength or weakness of their desired coffee and opens the valve allowing the coffee to be pumped into the bottle the user is ready to enjoy a cup of coffee at their destination or along the way to their destination of one embodiment.

### First HOCL Generator:

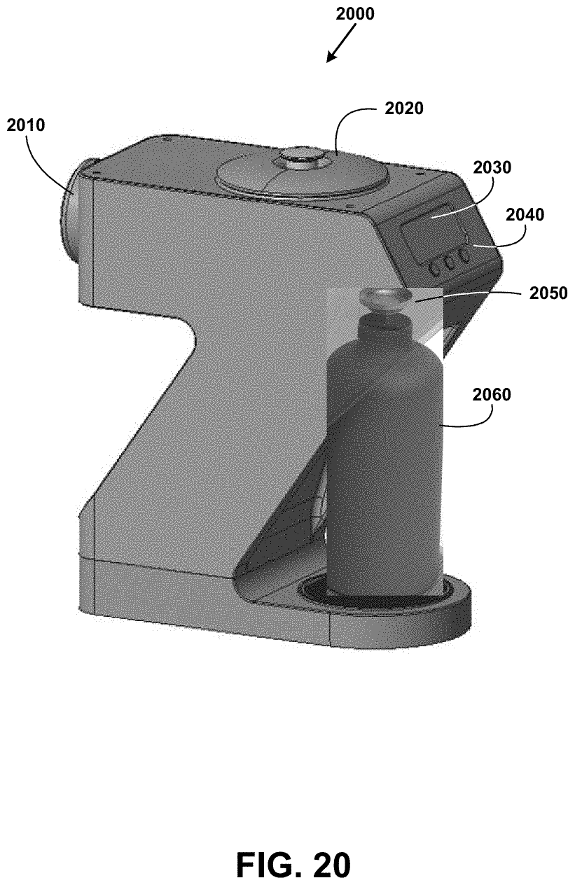

FIG. 20 shows for illustrative purposes only an example of a first HOCL generator of one embodiment. FIG. 20 shows a first HOCL generator 2000. The first HOCL generator 2000 is used to generate hypochlorous acid (HOCL) that is a non-toxic sanitizer for disinfecting surfaces and objects. HOCL destroys microorganisms including bacteria, viruses including Sars-Cov-2 that causes Covid-19 and fungi. The first HOCL generator 2000 includes an electrode attachment 2010. At least one electrode is used to perform electrolysis on a water solution of purified water and pure salt to create HOCL. The first HOCL generator 2000 includes a manual fill, salt cap with vent 2020. The cap is vented to release gases created during the electrolysis operation. An LCD control panel 2030 displays operations and status initiated using control buttons 2040. An HOCL dispenser 2050 is used to fill for example a portable container 2060 with the completed HOCL solution for use in applying the HOCL disinfectant onto surfaces of one embodiment.

In one embodiment the method is providing a first and a second hypochlorous acid generator for creating purified hypochlorous acid, automatically filling impure water into a mixing tank container of the hypochlorous acid generator, processing the impure water in the mixing tank container with exposure of ultraviolet light from a plurality of ultraviolet lights within the mixing tank container to purify the water, providing a plurality of control buttons coupled to an LCD control panel display for a user selecting the hypochlorous acid parts per million concentration targeted to be generated, operating a power current controller for regulating the electrical current level of at least one Double Cross Blade electrode coupled internally to the mixing tank container, determining an electrical current level and cycling period and duration of a phase pulsed current using an algorithm recorded in the power current controller, regulating the phase pulsed current using digital circuit controllers to produce an electrolysis operation, mixing the hypochlorous acid mixture using at least one rotation reversible impeller for circulating the hypochlorous acid mixture, and operating the first and second hypochlorous acid generator remotely using a hypochlorous acid generator app for transmitting and receiving operating signals on a user digital device hypochlorous acid generator settings. The first hypochlorous acid generator is configured for portable use for refilling hypochlorous acid application devices including hypochlorous acid spray and wipe-on device bottles. The second hypochlorous acid generator is configured for countertop use for refilling hypochlorous acid application devices including commercial and industrial motorized hypochlorous acid application devices. Pure salt is provided premeasured packages for adding pure salt to the purified water. Operations include automatically filling impure water into a mixing tank container of the hypochlorous acid generator using a flowmeter to measure filling volume. The second HOCL Generator can be placed on a countertop and any suitable support. Installing the hypochlorous acid generator app in a user digital device including a smart phone and is used to transmit predetermined control settings to a power current control and impeller speed control devices. Predetermined control settings are calculated using algorithms based on targeted parts per million settings for determining a current level setting, current timer setting and impeller speed control setting based on experimental results. A selected hypochlorous acid usage and algorithm calculated settings results are transmitted to a hypochlorous acid generator cloud for recording using hypochlorous acid generator app transmissions to a hypochlorous acid generator cloud. Accessing predetermined control settings based on usage including for food, disinfecting and professional following the FDA hypochlorous acid guidelines.

### First HOCL Generator Transparent View:

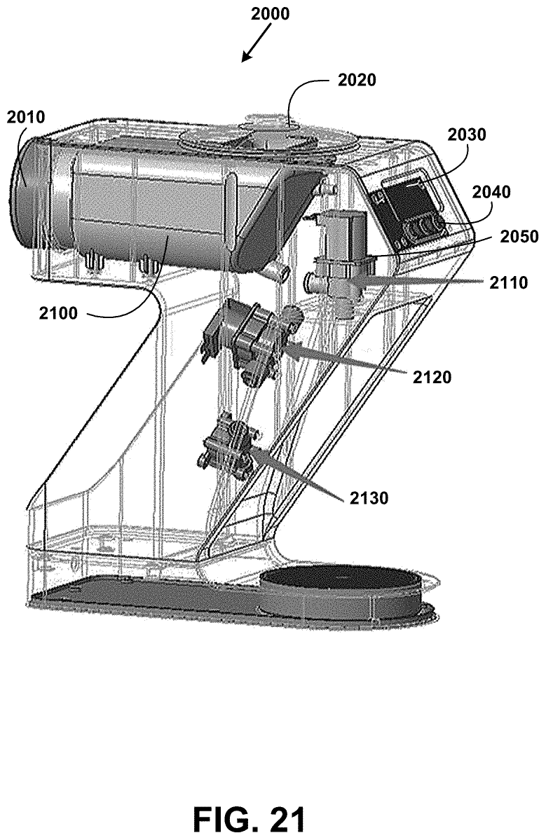

FIG. 21 shows for illustrative purposes only an example of a first HOCL Generator transparent view of one embodiment. FIG. 21 shows through the transparent view components on the interior of the first HOCL generator 2000. The first HOCL generator 2000 components include an electrode attachment 2010 the opening of the manual fill, salt cap with vent 2020, the LCD control panel 2030, control buttons 2040 and the HOCL dispenser 2050 of one embodiment.

### Second HOCL Generator Front:

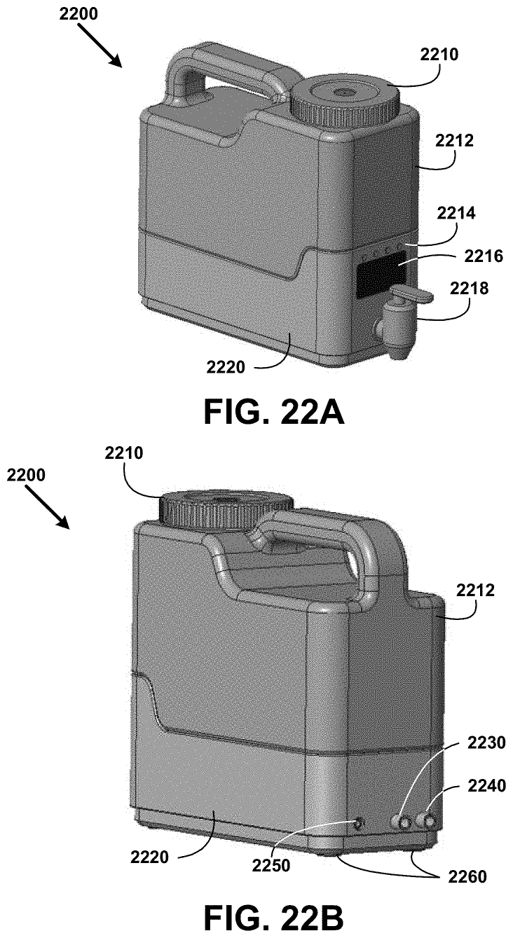

FIG. 22A shows for illustrative purposes only an example of a second HOCL generator front right side prospective view of one embodiment. FIG. 22A shows a second HOCL generator 2200 with a capacity to generate a large volume of HOCL. The second HOCL generator 2200 includes a vented fill cap 2210 for venting gases created during electrolysis. A mixing tank container 2212 is manually filled with purified water through the vented fill cap 2210 opening. Pure salt is also poured into the mixing tank container 2212 through the vented fill cap 2210 opening. A plurality of control buttons 2214 are used to selected and initiate operations that are displayed on a control panel 2216 on the front of a control base 2220 of one embodiment.

In another embodiment at least one hypochlorous acid generator for producing purified hypochlorous acid from purified water and pure salt, a mixing tank container hypochlorous acid generator for processing the purified water and pure salt, a cap with vent configured to release gases created during an electrolysis operation, a water intake port to fill the mixing tank container with fill water automatically using an automatic system intake valve, a water drain port to drain liquid from the mixing tank container, an AC port to route external power circuits connections to power the at least one hypochlorous acid generator, at least one crossing double electrode module configured to provide ultraviolet light to purify water and perform phase pulsed current electrolysis, an LCD control panel coupled to control buttons to display processing status and operation control settings for the electrolysis operation, a hypochlorous acid dispenser to refill hypochlorous acid application devices with purified hypochlorous acid, and a hypochlorous acid generator app on a user digital device to transmit hypochlorous acid generator control settings to a power current control and impeller speed control devices. The automatic system intake valve determines the total flow into the mixing tank container using a flow meter. The hypochlorous acid generator includes a control base to house the power current controller, LCD control panel, control buttons and control settings devices. The control base is configured for housing at least one digital device including at least one digital memory device, digital processor, a WIFI communication device. At least one ultraviolet light LED is coupled to the at least one crossing double electrode module.

### Second HOCL Generator Rear:

FIG. 22B shows for illustrative purposes only an example of a second HOCL generator rear left side prospective view of one embodiment. FIG. 22B shows in the rear left side view of the second HOCL generator 2200, vented fill cap 2210, mixing tank container 2212, control base 2220, water intake port 2230, water drain port 2240, AC port 2250 and rubber feet 2260. The water intake port 2230 provides a connection for filling the mixing tank container 2212 using a purified water supply including an automatic system intake valve. The automatic system intake valve determines the total flow into the mixing tank container 2212. Based on the mixing tank container 2212 volume and filled level using sensors for example a flow meter to measure the water level and automatic initiation of additional intake flow of purified water to a predetermined level. The AC port 2250 provides an entrance to the second HOCL generator 2200 to route an external power source to operate the elements of the second HOCL generator 2200 including electrolysis and control devices of one embodiment.

In yet another embodiment at least one hypochlorous acid generator is configured for producing purified hypochlorous acid from purified water and pure salt, a mixing tank container for containing the purified water and pure salt during processing, at least one crossing double electrode module configured to provide ultraviolet light to purify water and perform phase pulsed current electrolysis, a cap with vent configured to release gases created during an electrolysis operation, an impeller for mixing the purified water and pure salt during electrolysis, a hypochlorous acid dispenser to refill hypochlorous acid application devices with purified hypochlorous acid, and a hypochlorous acid generator app on a user digital device to transmit hypochlorous acid generator control settings to a power current control and impeller speed control devices. A water intake port coupled to the mixing tank container is configured to fill water automatically using an automatic system intake valve. A water drain port to drain liquid from the mixing tank container. An AC port is configured for routing external power circuit connections to power the at least one hypochlorous acid generator. An LCD control panel coupled to control buttons is configured to display processing status and operation control settings for the electrolysis operation.

### Mixing Tank Container Translucent View:

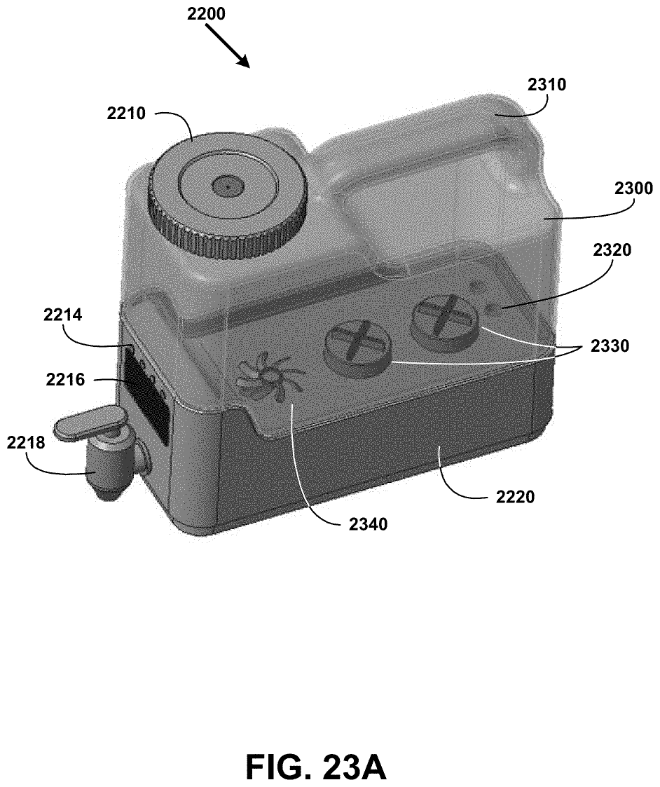

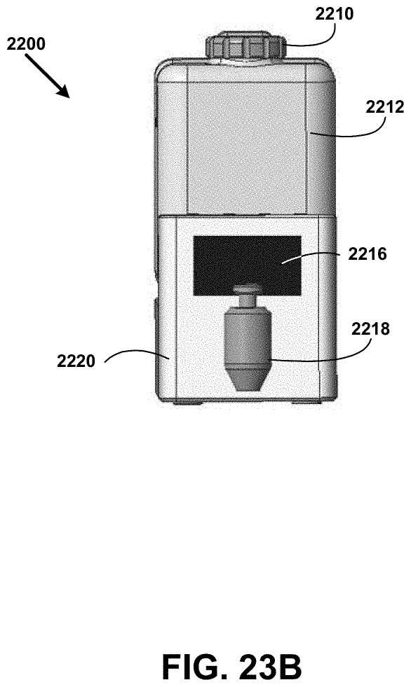

FIG. 23A shows for illustrative purposes only an example of a mixing tank container translucent prospective view of one embodiment. FIG. 23A shows the second HOCL generator 2200 with a mixing tank container translucent view 2300. The translucent view shows a handle 2310, water intake port interior view 2320, cross blade electrode module 2330, propeller impeller 2340 in the interior. The vented fill cap 2210, control buttons 2214, control panel 2216 and HOCL dispenser 2218 are also shown on the control base 2220 of one embodiment.

FIG. 23B shows for illustrative purposes only an example of a second HOCL generator front view of one embodiment. FIG. 23B shows the second HOCL generator 2200 vented fill cap 2210 and mixing tank container 2212. The control panel 2216 and HOCL dispenser 2218 are also shown on the control base 2220 of one embodiment.

### Cross Blade Electrode Module:

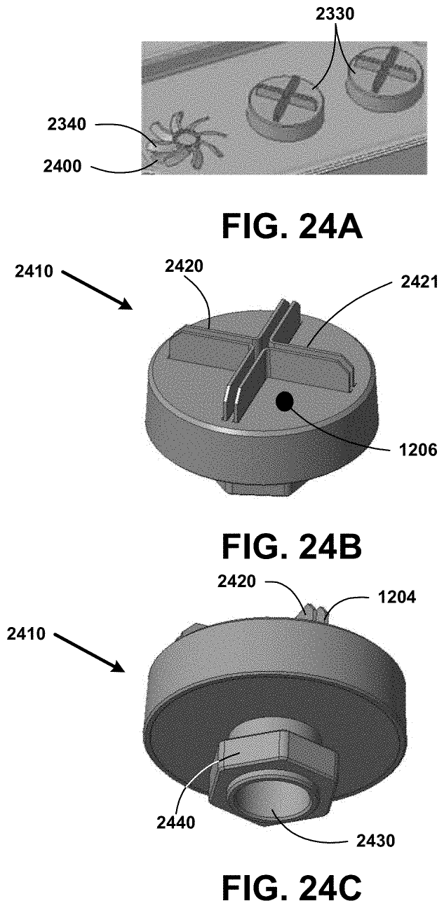

FIG. 24A shows for illustrative purposes only an example of a cross blade electrode module of one embodiment. FIG. 24A shows an interior view of the mixing tank container 2212 of FIG. 22A showing two of the cross blade electrode module 2330 units for performing electrolysis. Also showing is the propeller impeller 2340 for mixing the water solution during electrolysis for obtaining a uniform ppm. A mixing tank container drain 2400 is shown for draining the mixing tank container 2212 of FIG. 22A for example in a rinsing operation of one embodiment.

Ultraviolet LED:

FIG. 24B shows for illustrative purposes only an example of an ultraviolet LED of one embodiment. FIG. 24B shows a cross blade electrode module 2330 negative electrode 2421 and a positive electrode 2420. In one embodiment the cross blade electrode module 2330 includes a UV LED 1206 for projecting ultraviolet into the water solution for destroying microorganisms including bacteria, viruses including Sars-Cov-2 that causes Covid-19, fungi to sanitize the water solution of one embodiment.

### An Electric and Control Circuit Conduit:

FIG. 24C shows for illustrative purposes only an example of a cross blade electrode module bottom view of one embodiment. FIG. 24C shows the cross blade electrode module 2330 in a bottom prospective view. The cross blade electrode module 2330 includes a negative electrode 2421 and a positive electrode 2420. The power circuits to the electrodes are inserted through an electric and control circuit conduit 2430. The electric and control circuit conduit 2430 includes thread for securing a locking nut 2440 to hold the cross blade electrode module 2330 into position of one embodiment.

### A Power Current Controller:

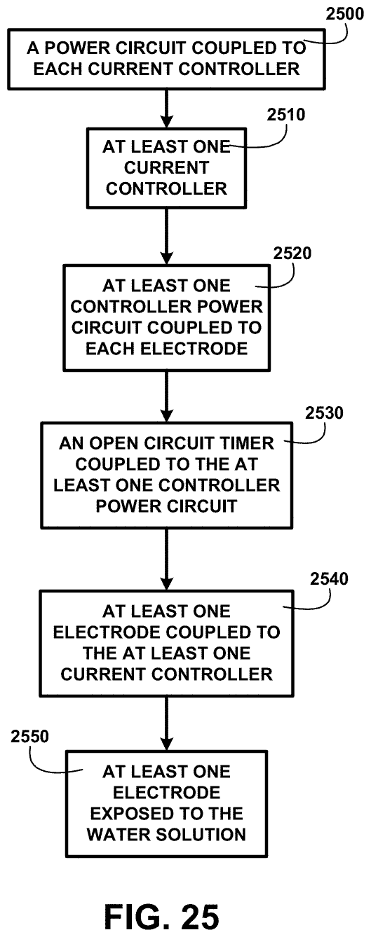

FIG. 25 shows a block diagram of an overview of a power current controller of one embodiment. FIG. 25 shows a power circuit coupled to each current controller 2500. At least one current controller 2510 is coupled to at least one controller power circuit coupled to each electrode 2520. An open circuit timer coupled to the at least one controller power circuit 2530 provides the predetermined current amp level to produce a targeted ppm in a predetermined time period. The predetermined current amp level is conducted to at least one electrode coupled to the at least one current controller 2540. At least one electrode exposed to the water solution 2550 provides electrolysis treatment to the water and salt solution to produce HOCL at the targeted ppm. The time period of the duration of electrolysis is reduced based on the number of electrode module assemblies being used of one embodiment.

### A HOCL Generator App:

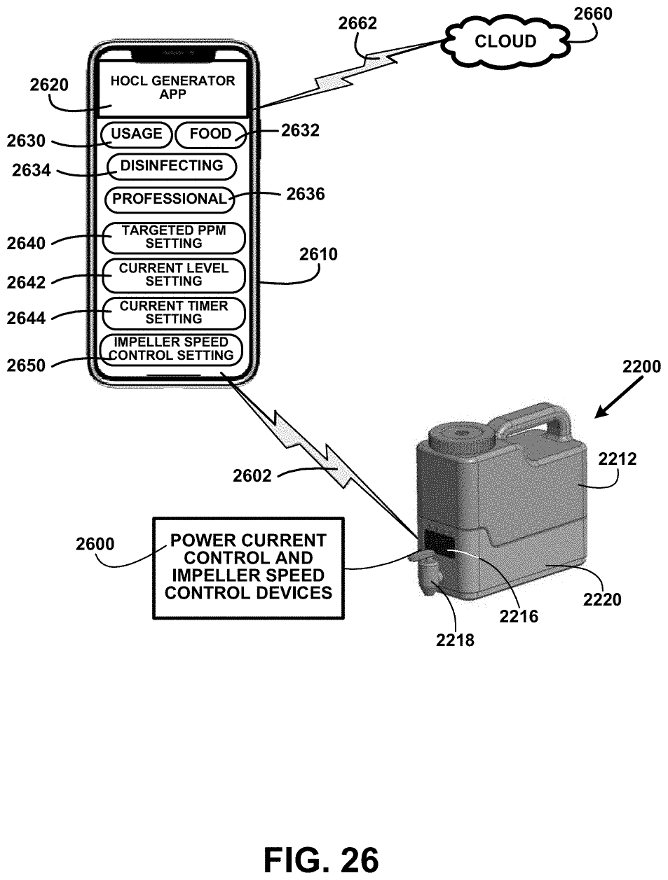

FIG. 26 shows for illustrative purposes only an example of a HOCL generator app of one embodiment. FIG. 26 shows the second HOCL Generator 2200 can be place on a countertop and any suitable support. The HOCL generated is filled into HOCL application devices for example a HOCL sprayer device. The second HOCL Generator 2200 includes the mixing tank container 2212, control panel 2216, control base 2220 and HOCL dispenser 2218.

In one embodiment a power current control and impeller speed control devices 2600 not shown is coupled to the second HOCL generator 2200. At least one second HOCL generator app transmission to second HOCL generator controller devices 2602 from a user digital device 2610 is receive to regulate the HOCL generation at a predetermined ppm. A second HOCL generator app 2620 installed in the user digital device 2610 for example a smart phone is used to transmit the predetermined control settings to the power current control and impeller speed control devices 2600.

The predetermined control settings are accessed in one embodiment based on usage 2630 including for food 2632, disinfecting 2634 and professional 2636 following the FDA HOCL guidelines. In another embodiment the predetermined control settings are calculated using algorithms based on a targeted ppm setting 2640. The algorithms display on the user digital device 2610 a current level setting 2642, current timer setting 2644 and impeller speed control setting 2650. The impeller speed control setting 2650 is determined based on the current timer setting 2644 and portable container volume of solution. In yet another embodiment the selected usage and algorithm calculated settings results are transmitted to a second HOCL generator cloud 2660 for recording using second HOCL generator app transmissions to the second HOCL generator cloud 2662.

The foregoing has described the principles, embodiments and modes of operation of the embodiments. However, the embodiments should not be construed as being limited to the particular embodiments discussed. The above described embodiments should be regarded as illustrative rather than restrictive, and it should be appreciated that variations may be made in those embodiments by workers skilled in the art without departing from the scope of the present invention as defined by the following claims.

What is claimed is:

1. A method, comprising:
providing a first and a second hypochlorous acid generator for creating purified hypochlorous acid;
automatically filling impure water into a mixing tank container of the hypochlorous acid generator;
processing the impure water in the mixing tank container with exposure of ultraviolet light from a plurality of ultraviolet lights within the mixing tank container to purify the water;
providing a plurality of control buttons coupled to an LCD control panel display for a user selecting the hypochlorous acid parts per million concentration targeted to be generated;
operating a power current controller for regulating the electrical current level of at least one Double Cross Blade electrode coupled internally to the mixing tank container;
determining an electrical current level and cycling period and duration of a phase pulsed current using an algorithm recorded in the power current controller;
regulating the phase pulsed current using digital circuit controllers to produce an electrolysis operation;
mixing the hypochlorous acid mixture using at least one rotation reversible impeller for circulating the hypochlorous acid mixture; and
operating the first and second hypochlorous acid generator remotely using a hypochlorous acid generator app installed on a user digital, for transmitting and receiving device hypochlorous acid generator operating settings signals.

2. The method of claim 1, wherein the first hypochlorous acid generator is configured for portable use for refilling hypochlorous acid application devices including hypochlorous acid spray and wipe-on device bottles.

3. The method of claim 1, wherein the second hypochlorous acid generator is configured for countertop use for refilling hypochlorous acid application devices including commercial and industrial motorized hypochlorous acid application devices.

4. The method of claim 1, further comprising pure salt in premeasured packages for adding pure salt to the purified water.

5. The method of claim 1, further comprising automatically filling impure water into a mixing tank container of the hypochlorous acid generator using a flowmeter to measure filling volume.

6. The method of claim 1, wherein the second HOCL Generator can be placed on a countertop and any suitable support.

7. The method of claim 1, further comprising installing the hypochlorous acid generator app in the user digital device including a smart phone is used to transmit predetermined control settings to a power current control and impeller speed control devices.

8. The method of claim 1, wherein predetermined control settings are calculated using algorithms based on targeted parts per million settings for determining a current level setting, current timer setting and impeller speed control setting based on experimental results.

9. The method of claim 1, further comprising a selected hypochlorous acid usage and algorithm calculated settings results are transmitted to a hypochlorous acid generator cloud for recording using hypochlorous acid generator app transmissions to a hypochlorous acid generator cloud.

10. The method of claim 1, further comprising accessing predetermined control settings based on usage including for food, disinfecting and professional following the FDA hypochlorous acid guidelines.

11. An apparatus, comprising:
at least one hypochlorous acid generator for producing purified hypochlorous acid from purified water and pure salt;
a mixing tank container hypochlorous acid generator for processing the purified water and pure salt;
a cap with vent configured to release gases created during an electrolysis operation;
a water intake port to fill the mixing tank container with fill water automatically using an automatic system intake valve;
a water drain port to drain liquid from the mixing tank container;
an AC port to route external power circuits connections to power the at least one hypochlorous acid generator;
at least one crossing double electrode module configured to provide ultraviolet light to purify water and perform phase pulsed current electrolysis;
an LCD control panel coupled to control buttons to display processing status and operation control settings for the electrolysis operation;
a hypochlorous acid dispenser to refill hypochlorous acid application devices with purified hypochlorous acid; and
a hypochlorous acid generator app installed on a user digital device to transmit hypochlorous acid generator control settings to a power current control and impeller speed control devices.

12. The apparatus of claim 11, wherein the automatic system intake valve determines the total flow into the mixing tank container using a flow meter.

13. The apparatus of claim 11, further comprising a control base to house the power current controller, LCD control panel, control buttons and control settings devices.

14. The apparatus of claim 11, further comprising at least one ultraviolet light LED coupled to the at least one crossing double electrode module.

15. The apparatus of claim 11, further comprising a control base for housing at least one digital device including at least one digital memory device, digital processor, a WIFI communication device.

16. An apparatus, comprising:
at least one hypochlorous acid generator for producing purified hypochlorous acid from purified water and pure salt;
a mixing tank container for containing the purified water and pure salt during processing;
at least one crossing double electrode module configured to provide ultraviolet light to purify water and perform phase pulsed current electrolysis;
a cap with vent configured to release gases created during an electrolysis operation;
an impeller for mixing the purified water and pure salt during electrolysis;
a hypochlorous acid dispenser to refill hypochlorous acid application devices with purified hypochlorous acid; and
a hypochlorous acid generator app installed on a user digital device to transmit hypochlorous acid generator control settings to a power current control and impeller speed control devices.

17. The apparatus of claim 16, further comprising a water intake port coupled to the mixing tank container to fill water automatically using an automatic system intake valve.

18. The apparatus of claim 16, further comprising a water drain port to drain liquid from the mixing tank container.

19. The apparatus of claim 16, further comprising an AC port to route external power circuits connections to power the at least one hypochlorous acid generator.

20. The apparatus of claim 16, further comprising an LCD control panel coupled to control buttons to display processing status and operation control settings for the electrolysis operation.
